(19) United States
(12) Patent Application Publication
(10) Pub. No.: US 2015/0104840 A1
(43) Pub. Date: Apr. 16, 2015

(54) METHOD OF CONTINUOUS FERMENTATION PROCESS FOR SUCCINIC ACID BY MICROBIAL CELLS OF ACTINOBACILLUS SUCCINOGENES

(71) Applicant: KANGWON NATIONAL UNIVERSITY University-Industry Cooperation Foundation, Chuncheon-si (KR)

(72) Inventors: Gie-Taek CHUN, Chuncheon-si (KR); Sang-Min PARK, Gangneung-si (KR); Dohoon LEE, Seoul (KR); Sangyong KIM, Cheonan-si (KR); Kyu-Ri EUM, Gapyeong-gun (KR)

(73) Assignee: KANGWON NATIONAL UNIVERSITY University-Industry Cooperation Foundation, Chuncheon-si (KR)

(21) Appl. No.: 14/155,618

(22) Filed: Jan. 15, 2014

(30) Foreign Application Priority Data
Oct. 15, 2013 (KR) 10-2013-0122726
Oct. 15, 2013 (KR) 10-2013-0122727
Oct. 15, 2013 (KR) 10-2013-0122728

(51) Int. Cl. C12P 7/46

(52) U.S. Cl. CPC C12P 7/46
USPC 435/145; 435/289.1

(57) ABSTRACT

The present invention relates to a continuous fermentation process using Actinobacillus succinogenes. It was confirmed that in a cell recycled process, a production amount of succinic acid was about 60 g/L and productivity was about 3.873 g/L per hour. It was confirmed that a cell recycled fermentation process was increased in amount of microbial cells by about 2 times or more, increased in production amount of succinic acid by about 5 times, and increased in productivity of succinic acid by about 8 times or more as compared with the typical continuous culture. Such a succinic acid production process with high productivity and high yield rate can reduce production cost and can also produce succinic acid on an industrial level even at a pilot-scale culture unit without scaling up a culture unit. Therefore, if the process of the present invention is applied, it is expected to be more advantageous for industrial application.

### CROSS-REFERENCE TO RELATED APPLICATION

This application claims priority to and the benefit of Korean Patent Application No. 2013-0122726, filed Oct. 15, 2013, 2013-0122727, filed Oct. 15, 2013 and 2013-0122728, filed Oct. 15, 2013, the disclosure of which is incorporated herein by reference in its entirety.

### BACKGROUND

1. Field of the Invention

The present invention relates to a continuous fermentation process using Actinobacillus succinogenes, and more particularly, to a continuous fermentation process that employs bacteria recycled system using an optimal culture medium, a drip tube, and a cell separator, thereby stabilizing a system and producing succinic acid with high efficiency.

2. Discussion of Related Art

Succinic acid is a linear chain dicarboxylic acid with structural formula HOOC—(CH₂)₂—COOH. Sodium salt has a taste component of shellfish and is used as a flavor enhancer. The succinic acid is also referred to as amber acid based on the record that it was obtained from amber as fossil resin by means of distillation by R. Agricola in 1550. The succinic acid is formed of colorless columnar or plate crystals and has the molecular weight of 118.09, the melting point of 185° C., the boiling point of 235° C., and the specific gravity of 1.564. Further, the succinic acid is a major organic acid constituting TCA cycle and is one of carboxylic acids. During a step for producing the succinic acid in the TCA cycle, alpha ketoglutaric acid is dehydrogenated and decarboxylated to form a succinyl Co-A and converted again to the succinic acid, and the succinic acid is oxidized to fumaric acid by a succinic dehydrogenase.

Among succinic acids which can be produced by chemical synthesis methods and microbial fermentation with microorganisms, only succinic acids in a small amount used for special purposes such as an additive or a preserving agent for drugs and foods have been produced by a microbial fermentation method. Meanwhile, most succinic acids used industrially have been synthesized from n-butane and acetylene derived from crude oil or a liquefied natural gas by the big chemical companies in the U.S., Europe, Japan, and China. Typically, a method for chemically synthesizing a succinic acid has a problem that harmful solid waste, waste liquid, and waste gas (including carbon monoxide) are discharged in a large amount, and uses highly exhaustible fossil fuel, which is highly exhaustible, as a base material. Therefore, a method for producing a succinic acid using microorganisms urgently needs to be researched and developed as an alternative method thereof. Further, since production cost continues to increase due to a rise in oil prices, an alternative production process is urgently needed and a method for biologically producing a succinic acid from grain has become an object of attention.

A succinic acid is a C₄ organic acid which can be applied to various fields related to drugs, foods, and petrochemical processes and was selected as one of 10 important materials by the United States Department of Energy. Recent advances in fermentation technology have reached a level sufficient to replace a conventional production process using substitution of hydrogen, and, thus, a lot of research has been carried out worldwide. Further, the international market worth 20 trillion won or more a year is created, and, thus, a worth of the succinic acid is very high.

Culture types can be classified into a general aerobic fermentation and anaerobic fermentation represented by alcoholic fermentation depending on a characteristic of a microorganism, can also be classified into liquid culture and solid culture depending on a state of a culture medium, and can be operationally classified into batch culture and continuous culture.

A batch culture is a kind of closed reaction in which culture is continued by using a bioreactor until culture medium components are decreased as the culture proceeds and a main substrate is completely consumed, and microorganisms proliferate and a target product is accumulated, and the batch culture is the most common method in the fermentation industry. The batch culture includes five phases of a lag phase, a logarithmic growth phase, a steady phase, a deceleration phase, and a stationary phase after inoculation. When a cell is cultured, the cell in a steady phase is inoculated into a new culture medium and then a growth cycle is repeated. If a cellular environment is not uniform due to a change in culture medium components or cell density during a culture process, productivity is low. However, a batch culture apparatus or method is commonly used since it is easy to use as compared with the continuous batch.

A fed-batch culture is a culture method in which a substrate as a proliferation limiting factor is continuously supplied little by little and maintained at a constant low concentration, and in the fed-batch culture, an environment can be continuously regulated to be suitable for culture. Typically, the fed-batch culture is used to control a feeding rate in the case of using a proliferation limiting substrate as a base material or to prevent feedback control by controlling supply of an auxotrophic material in the case where productivity is lowered by a control mechanism such as inhibition of catabolite or an auxotrophic variant is used when a concentration of the substrate is increased. However, in the fed-batch culture, it is difficult to regulate an optimum condition or to maintain an adequate feeding rate, and, thus, a dilution rate can be sharply decreased. Therefore, it is difficult to control a culture process.

A continuous culture is a culture method in which a culture fluid is continuously supplied to a bioreactor and a culture fluid in the same amount is discharged from the bioreactor so as to culture microorganisms while maintaining a steady state. Chemostat is a method for controlling proliferation of microorganisms using a carbon source or a nitrogen source of a nutrient broth as a proliferation limiting factor. Turbidostat is a method for continuously supplying and discharging a batch so as to maintain a concentration of microorganisms.

In the continuous culture, when a steady state of culture is established, a reaction is carried out in the steady state, and after the steady state, concentrations of a cell, a product, and a substrate are constantly maintained. Further, automatic control, standardization and automation of operation are practicable, and a labor-saving effect can be obtained. The greatest advantage of the continuous culture is high productivity caused by extension of a time for a steady phase and minimization of time for a lag phase and a stationary phase.

However, the greatest advantage of the current continuous culture process is that a cell specific growth velocity can be artificially fixed by regulating a speed of a liquid supplied with a pump. In addition to this, the present invention further includes a process of adding a small amount of carbonate ion concentrate in a fed-batch manner by using physiological characteristics of a producing strain to overcome a critical dilution rate and increase a production amount of succinic acid. Further, a fermentation process by means of continuous culture is a small-scale process as compared with a batch culture but produces as much products as the batch culture. Therefore, a scale of a bioreactor can be reduced efficiently, resulting in a reduction in production cost. Accordingly, industrial competitiveness in the market can be obtained.

However, the continuous culture is a process for producing only one kind of product, and a specific growth velocity of an infectious microbe is typically higher than a specific growth velocity of a producing strain. Therefore, if contaminated with an infectious microbe, a producing strain can be substituted with the infectious microbe. As for an improved strain, a genetic character may be modified and a revertant may be created. If a specific growth velocity of the revertant is high, a production amount is greatly reduced. Further, in terms of process cost, cost for separation and recovery can be increased due to a low concentration of a product.

Further, productivity of the batch culture or the fed-batch culture is low in a standard scale bioreactor. That is, as described above, the batch culture or the fed-batch culture includes several processes and thus it is not efficient. Therefore, in order to solve the problem, the continuous culture is employed and high productivity can be obtained. However, in the case of using a method of immobilizing microorganisms, clogging may occur due to overexpression of the microorganisms.

Therefore, most fermentation processes use suspension instead of immobilization to manufacture products. However, in the case where continuous culture is carried out by means of suspension culture, there is a limit in increasing productivity due to washout of a microbial cell.

Meanwhile, a reproduction rate (dX/dt) of microbial cells in a unit volume of a reactor can be expressed by the following equation.

dX/dt=D(X1−X0)+(μ−kd)X

Herein, D represents a dilution rate, X represents a concentration of the microbial cells, X0 represents a concentration of the microbial cells introduced into the reactor, X1 represents a concentration of the microbial cells discharged from the reactor, μ represents a production rate of the microbial cells, and kd represents a death rate. In this case, in order to prevent washout of the microbial cells, (μ−kd)X should be greater than D(X1−X0). However, since the productivity is in proportion to DX, if the operation is carried out at low D in order to prevent washout of the microbial cells, the productivity is lowered. Therefore, in order to improve the productivity, various methods of collecting microbial cells from a flow discharged from a reactor have been designed.

Methods of collecting cells include a precipitation method and a filtration method which uses hollow fibers. Currently, the two methods are all used in the treatment of municipal sewage, but are not used in biological processes due to low permeation rate and the phenomenon that microorganisms adhere to the surface of a membrane, respectively. In this case, in order to increase productivity, a continuous operation should be carried out while increasing “DX” of the above equation, but continuous high-concentration culture has not been realized due to the above-described reasons.

A cell separator is an apparatus used for cell recycle during continuous culture and is configured to collect microbial cells from a culture fluid discharged to the outside and use the microbial cells for fermentation. Since the microbial cells are collected, a high dilution rate can be maintained even at a low cell specific growth velocity and productivity becomes maximized overall. An effect of such an apparatus is remarkable in the continuous culture, and apparatuses using a membrane or a filter have been widely used to culture bacteria.

However, when a cell separator using a membrane or a filter is used, if a culture fluid has viscosity, the culture fluid and microbial cells may clog or damage the membrane or the filter. Therefore, it is not effective, and separation efficiency is sharply decreased.

Korean Patent No. 10-0301960

### SUMMARY OF THE INVENTION

An object of the present invention is to provide a continuous culture method for mass production of succinic acid using Actinobacillus succinogenes, the continuous culture method comprising: continuously supplying a culture medium comprised of glucose, yeast extract, and corn steep liquor, and sodium hydrogen carbonate (NaHCO₃) to a bioreactor; and continuously removing a culture fluid from the bioreactor.

Another object of the present invention is to provide a continuous culture system for mass production of succinic acid using Actinobacillus succinogenes, the continuous culture system comprising a bioreactor in which a culture fluid is stored; a cell separator configured to separate a culture fluid supplied to the bioreactor into microbial cells and the culture fluid by using a separation means and discharge microbial cells and the culture fluid; and a drip tube configured to collect a culture medium supplied from a culture medium supply unit and the microbial cells discharged from the cell separator and supply the culture medium and the microbial cells to the bioreactor.

In order to achieve the above-described objects, the present invention provides a continuous culture method for mass production of succinic acid using Actinobacillus succinogenes, the continuous culture method comprising: continuously supplying a culture medium comprised of glucose, yeast extract, and corn steep liquor, and sodium hydrogen carbonate (NaHCO₃) to a bioreactor; and continuously removing a culture fluid from the bioreactor.

In an exemplary embodiment of the present invention, the continuous culture method may further comprise: separating the Actinobacillus succinogenes from the culture liquid continuously removed; and supplying the separated Actinobacillus succinogenes to the bioreactor.

In an exemplary embodiment of the present invention, the continuous culture method may further comprise: supplying magnesium carbonate (MgCO₃) having a concentration of 10 to 30 g/L at a velocity of 2 to 4 ml/hr.

In an exemplary embodiment of the present invention, the culture medium may be supplied at a velocity of 1 to 100 ml/hr and the sodium hydrogen carbonate (NaHCO₃) having a concentration of 8 to 10 g/L may be supplied at a velocity of 2 to 4 ml/hr.

In an exemplary embodiment of the present invention, the continuous culture method may aerate and supply carbon dioxide at a speed of 0.4 to 0.8 vvm.

In an exemplary embodiment of the present invention, the glucose may be contained at a concentration of 45 to 65 g/L, the yeast extract may be contained at a concentration of 5 to 8 g/L, and the corn steep liquor may be contained at a concentration of 1 to 14 g/L.

In an exemplary embodiment of the present invention, the Actinobacillus succinogenes may be a strain UK13 (KCTC 12233BP).

In an exemplary embodiment of the present invention, the culture medium, the sodium hydrogen carbonate, and the magnesium carbonate may be supplied at a velocity of 1 to 100 ml/hr.

In an exemplary embodiment of the present invention, the continuous culture method may further comprise: increasing a concentration of microbial cells by supplying the continuously removed culture fluid to the bioreactor at a velocity of 15 to 22.5 ml/hr.

In an exemplary embodiment of the present invention, the step of separating the Actinobacillus succinogenes may be carried out by using a cell separator.

In an exemplary embodiment of the present invention, the culture medium, the sodium hydrogen carbonate, or the magnesium carbonate may be supplied by using a drip tube.

Further, the present invention provides a continuous culture system for mass production of succinic acid using Actinobacillus succinogenes, the continuous culture system comprising: a bioreactor in which a culture fluid is stored; a cell separator configured to separate a culture fluid supplied to the bioreactor into microbial cells and the culture fluid by using a separation means and discharge the microbial cells and the culture fluid; configured to prevent contamination of a culture medium supplied from a culture medium supply unit and supply the culture medium to the bioreactor while controlling a supply rate of the culture medium; and a collection line configured to collect the microbial cells discharged from the cell separator and supply the microbial cells to the bioreactor.

In an exemplary embodiment of the present invention, the cell separator may have a predetermined size and includes an upper vertical portion formed at an upper part, a culture fluid outlet having a smaller diameter than the upper vertical portion and provided under the upper vertical portion with a space between them, and an inclined portion may be formed between the upper vertical portion and the culture fluid outlet.

In an exemplary embodiment of the present invention, the cell separator may have a predetermined size and includes an upper inclined portion formed at an upper part, a culture fluid outlet having a smaller diameter than the upper inclined portion and provided under the upper inclined portion with a space between them, and an upper vertical connection portion and a lower inclined connection portion may be continuously formed between the upper inclined portion and the culture fluid outlet.

In an exemplary embodiment of the present invention, an inner surface of the cell separator may include a curved surface guiding portion that allows the culture fluid to be rapidly discharged through the culture fluid outlet.

In an exemplary embodiment of the present invention, the separation means may be one of a cylindrical tube or a filter comprised of multiple mesh layers.

In an exemplary embodiment of the present invention, the drip tube may include a tube connection portion connected to the culture medium supplying unit and a speed control unit configured to control a supply rate of the culture medium.

In an exemplary embodiment of the present invention, at one side of the drip tube, a carbon dioxide outlet may be formed to discharge carbon dioxide supplied along with the microbial cells to the outside or a predetermined position.

If succinic acid is produced by continuous culture according to the present invention, high productivity of succinic acid as compared with the batch culture, and, thus, a production process can be carried out at a low cost as compared with the conventional production cost. Further, through industrial scale liquid culture using the culture medium for liquid culture of a strain for producing succinic acid and the continuous culture process according to the present invention, it is possible to supply succinic acid, which can be used in various fields such as petrochemical derivatives and drugs, and the food industry, in large amount with competitiveness at a low cost.

Furthermore, in the case of using the drip tube and the cell separator of the present invention, it is possible to prevent contamination during fermentation and increase a concentration of microbial cells. Therefore, a production amount of succinic acid can be increased together with an increase in production yield rate, and, thus, high economic efficiency can be achieved through a process minimizing use of a culture medium.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is a graph comparing an amount of microbial cells between when a continuous culture fermentation process added with magnesium carbonate in a fed-batch manner is carried out and when continuous culture is carried out;

FIG. 2 is a graph comparing an amount of residual glucose between when a continuous culture fermentation process added with magnesium carbonate in a fed-batch manner is carried out and when continuous culture is carried out;

FIG. 3 is a graph comparing a production amount of succinic acid between when a continuous culture fermentation process added with magnesium carbonate in a fed-batch manner is carried out and when continuous culture is carried out;

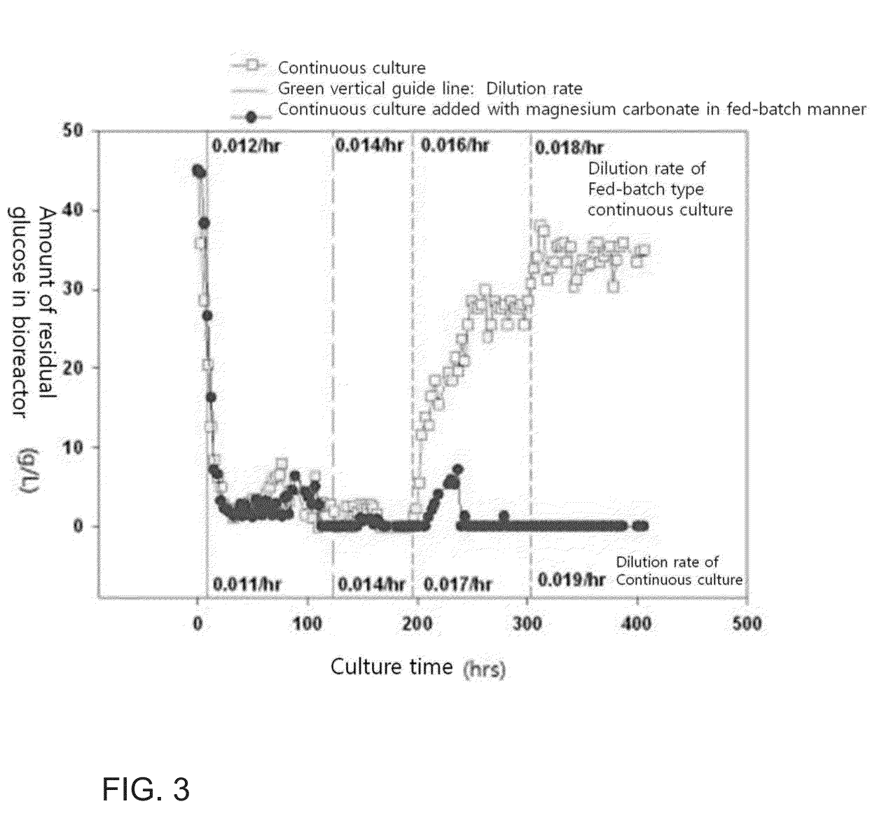

FIG. 4 is a graph comparing a measured amount of microbial cells with respect to glucose between when a continuous culture fermentation process added with magnesium carbonate in a fed-batch manner is carried out and when continuous culture is carried out;

FIG. 5 is a graph comparing a measured amount of succinic acid with respect to glucose between when a continuous culture fermentation process added with magnesium carbonate in a fed-batch manner is carried out and when continuous culture is carried out;

FIG. 6 is a graph comparing a production amount of a production amount of succinic acid with respect to an amount of microbial cells between when a continuous culture fermentation process added with magnesium carbonate in a fed-batch manner is carried out and when continuous culture is carried out;

FIG. 7 is a schematic diagram illustrating a continuous fermentation process;

FIG. 8 is a graph comparing an amount of microbial cells between when a cell recycled fermentation process is carried out and when continuous culture is carried out;

FIG. 9 is a graph comparing an amount of residual glucose between when a cell recycled fermentation process is carried out and when continuous culture is carried out;

FIG. 10 is a graph comparing a production amount of succinic acid between when a cell recycled fermentation process is carried out and when continuous culture is carried out;

FIG. 11 is a graph comparing a yield rate of microbial cells with respect to glucose between when a cell recycled fermentation process is carried out and when continuous culture is carried out;

FIG. 12 is a graph comparing a yield rate of succinic acid with respect to glucose between when a cell recycled fermentation process is carried out and when continuous culture is carried out;

FIG. 13 illustrates culture when a cell recycled fermentation process is actually carried out;

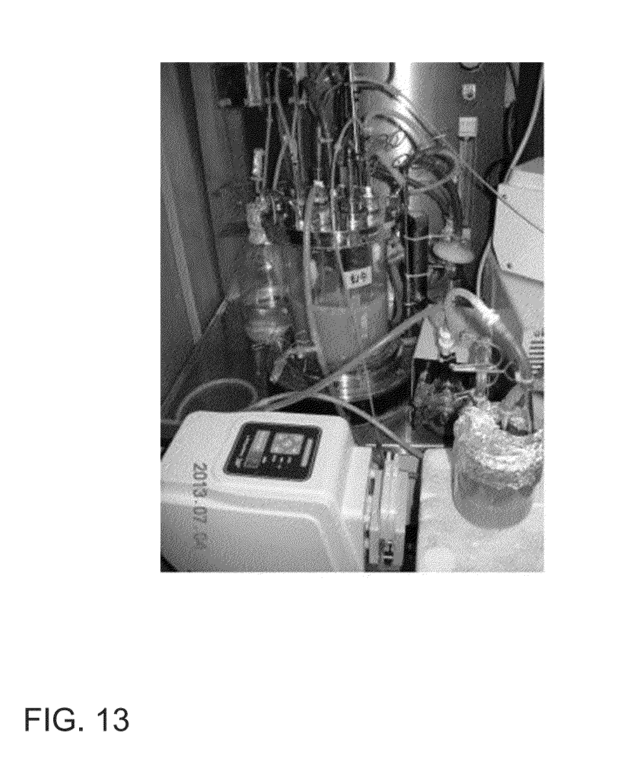

FIG. 14 is a configuration view of a cell recycled system according to the present invention;

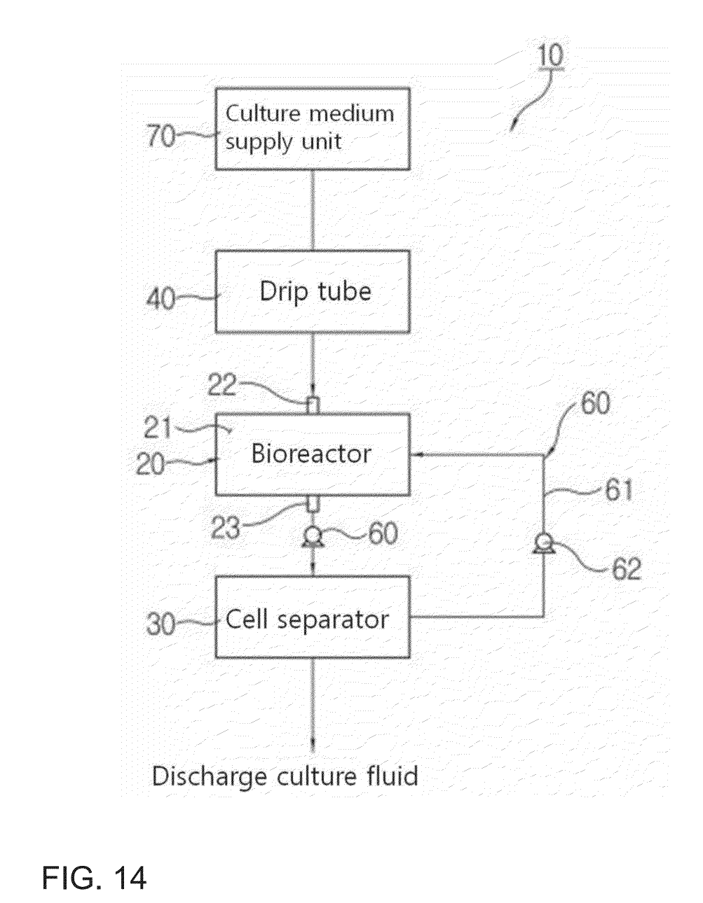

FIGS. 15(a) to 15(c) illustrate an example of a cell separator constituting the cell recycled system according to the present invention;

FIG. 16 is a photo showing an example of the cell separator constituting the cell recycled system according to the present invention;

FIGS. 17(a) to 17(d) illustrate a drip tube constituting the cell recycled system according to the present invention;

FIG. 18 is a graph showing cell growth in a continuous culture process repeated several times; and

FIG. 19 is a graph showing a yield rate of microbial cells with respect to glucose and a yield rate of succinic acid with respect to glucose by using information of an amount of microbial cells, an amount of residual glucose, and a production amount of succinic acid.

### EXPLANATION OF CODES

10: Cell recycled system, 20: Bioreactor, 30: Cell separator, 40: Drip tube, 50: Separation means, 60: Collection line

### DETAILED DESCRIPTION OF EXEMPLARY EMBODIMENTS

The terms used in the present invention will be defined as follows.

Through the present specification, the term “comprises or includes” and/or “comprising or including” means that one or more other components, steps, operation and/or existence or addition of elements are not excluded in addition to the described components, steps, operation and/or elements unless context dictates otherwise.

The term “continuous culture” used herein refers to a fermentation method including continuous supply of nutrients, supply of a substrate, and production of cells in a bioreactor (bioreactor). Such continuous supply, removal, or production of cells may be carried out in the same stream or different streams. A continuous process causes achievement of a steady state in the bioreactor. The term “steady state” means that all of measurable variables (i.e. a supply rate, concentrations of the substrate and the nutrients maintained in the bioreactor, a concentration of cells in the bioreactor and removal of cells from the bioreactor, removal of a product from the bioreactor, and conditional variables such as a temperature and a pressure) are constant over time.

All technical terms used in the present invention have the usual meaning conventionally understood by one of ordinary skill in the art to which this invention pertains, unless context defines otherwise. Further, although preferable methods and materials are described in the present specification, those similar or equivalent to the methods and materials fall within the scope of the present invention. All publications cited in the present specification as reference documents are incorporated herein by reference in their entirety.

As an effective fermentation process for producing succinic acid, a continuous culture process is known. Unlike the batch culture, the continuous culture process is a fermentation process in which a sterilized culture medium is continuously supplied to a bioreactor and a fermentation culture fluid including microorganisms and a target material mixed therein is discharged to the outside of the bioreactor as much as supplied so as to ferment the microorganisms while maintaining a constant liquid amount in the bioreactor. It is also referred to as “chemostat” because a stoichiometric characteristic in a culture fluid is constant in a steady state.

Actinobacillus succinogenes is a facultative anaerobe, and as a kind of rumen bacteria, the Actinobacillus succinogenes is one of the most remarkable bacteria for producing high-concentration succinic acid.

The present invention relates to a culture medium composition optimized for a cell recycling continuous system for mass-producing succinic acid through continuous culture using Actinobacillus succinogenes as a producing strain, and a cell recycle method. In particular, conditions for mass production of succinic acid are established by supplying magnesium carbonate (MgCO₃) and/or sodium hydrogen carbonate (NaHCO₃) in a fed-batch manner, which facilitates continuous production of succinic acid.

The present invention relates to an invention capable of producing succinic acid more stably with a high yield rate by using a drip tube and a cell separator when succinic acid is produced by means of a continuous culture process, and to be more specific, to a drip tube capable of preventing contamination of Actinobacillus succinogenes grown at a high growth velocity into a culture medium tank and an invention capable of facilitating a reuse of cells discarded by effectively separating cells during continuous culture and thus greatly increasing an amount of Actinobacillus succinogenes. To be still more specific, both the drip tube and the cell separator are configured to separate solids and cells from a supernatant by using a density difference caused by gravity, and the drip tube is provided on an inlet for an inflow from the sterilized culture medium tank to the bioreactor and configured to prevent a reverse flow from the bioreactor to the sterilized culture medium tank.

Typically, in the continuous culture process in which a non-used culture medium is continuously supplied and a culture fluid is discharged, microorganisms such as Actinobacillus succinogenes grown at a very high growth velocity may adhere to and grow at a culture medium inlet by bubbles generated during fermentation or an aerated gas, and if an inflow velocity of the culture medium is slow, the microorganisms enter a sterilized and non-used medium tank through a culture medium inlet line and contaminate the tank. If the drip tube is provided at this culture medium inlet line of the bioreactor, it is possible to effectively prevent such a phenomenon.

The cell separator of the present invention is configured to separate solids from a supernatant by using a density difference caused by gravity, and particularly, includes a separation tube therein, and, thus, efficiency of separation of solids even from a fluid having a high flow velocity and high viscosity can be improved. Therefore, efficiency of the continuous culture can be maximized.

Hereinafter, the present invention will be explained in detail with reference to Examples. However, the following Examples are provided for illustration of the present invention more specifically, but do not limit the scope of the present invention.

<Materials and Apparatuses>

Glucose used in the present invention was purchased from Daejung Co., Ltd., sodium hydrogen carbonate was purchased from Duksan Inc., and yeast extract, corn steep liquor solids, and magnesium carbonate were purchased from Sigma Aldrich.

### EXAMPLE 1

1. Producing Strain

As a succinic acid producing strain according to the present invention, a strain UK13 (KCTC 12233BP), as a mutant, genetically modified from Actinobacillus succinogenes (ATCC 55618) purchased from ATCC (American Type Culture Collection) was used.

2. Preservation of Strain

The present inventors preserved the producing strain and then divided and used a certain amount thereof. A culture medium for preserving the producing strain according to the present invention was a TSA (tryptic soy agar) medium (15 g of pancreatic digest of casein, 5 g of papaic digest of soybean, 5 g of NaCl, 15 g of agar, and 1 l of distilled water) as a solid culture medium.

The preserved strain was used by preserving microbial cells obtained through liquid culture at 4° C. or in a 20% glycerol stock at −80° C., and if necessary, taking out the preserved stock, inoculating the stock into a solid passaged culture medium, and culturing the stock.

3. Growth Culture (or Starter Culture) and Inoculation

The present inventors carried out growth culture in order to increase an amount of microbial cells before production culture of succinic acid. Through the growth culture using a TSB (tryptic soy broth) medium (17 g of pancreatic digest of casein, 3 g of papaic digest of soybean, 2.5 g of dextrose, 5 g of NaCl, 2.5 g of K₂HPO₄ (potassium phosphate dibasic), and 1 l of distilled water), microbial cells having high activity were obtained.

A single colony grown at a solid culture medium was aseptically collected and inoculated into a liquid culture medium to be 1% (v/v). Culture was carried out in a 2.5 L stirred tank reactor with a culture volume of 1.2 L. Growth culture of initial microbial cells was carried out in a shaking incubator at 38° C. at 200 rpm for 1 to 2 days, and liquid growth culture was carried out in a glass tube having a volume of 50 ml with an operation volume of 5 ml and the glass tube was tilted for smooth culture. A primary growth culture was cultured for 12 to 15 hours, and a secondary growth culture was cultured in a 250 ml flask with an operation volume of 30 ml for 6 to 12 hours, and then inoculated into a bioreactor for production of a target material.

4. Continuous Culture Method

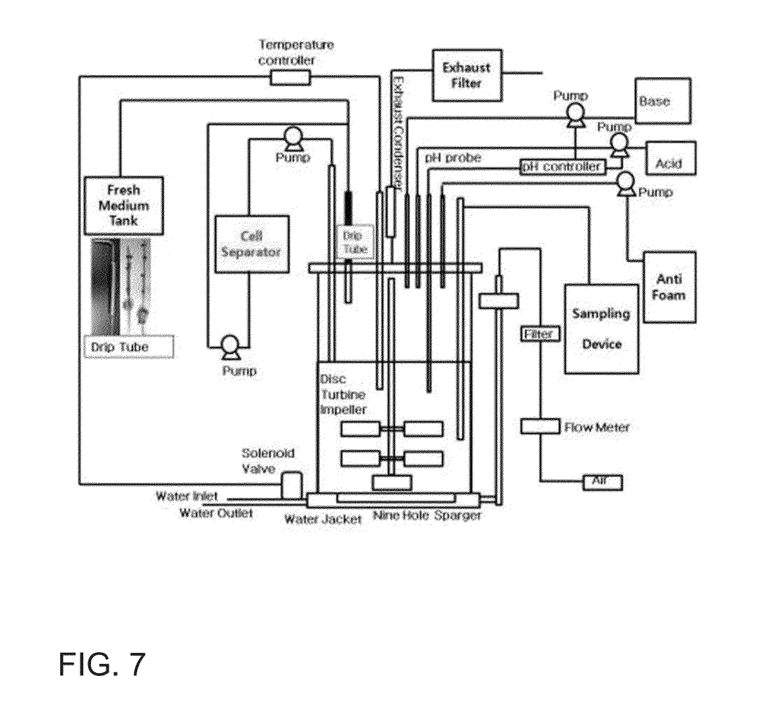

Continuous culture for mass-producing succinic acid by using a strain, Actinobacillus succinogenes, according to the present invention was as shown in a schematic diagram of FIG. 7.

According to the present invention, microorganisms can be cultured by a culture method such as a continuous culture method, a fed-batch culture method, or a combination of the continuous culture method and the fed-batch culture method with a culture system such as a multi stage system or a cell recycled system, but not limited thereto.

To be specific, in an example of the present invention, a liquid culture medium was put into a bioreactor and a producing strain in an amount of 1% was inoculated into a culture tank, and then continuous culture was carried out. Magnesium carbonate and/or sodium hydrogen carbonate was additionally supplied to a production medium in a fed-batch manner. Further, the culture was carried out with a cell recycled system to maintain a density of cells, and the Actinobacillus succinogenes was filtered, washed, and collected to be supplied again to the culture tank.

5. Culture Medium and Culture Conditions

In both of the growth culture and the production culture according to the present invention, the strain in an amount of 1% was inoculated, and as a production medium, the production medium including 45 μl of glucose, 10 μl of yeast extract, 10 μl of corn steep liquor, 5 μl of sodium hydrogen carbonate, and 20 μl of magnesium carbonate (MgCO₃) was used. In the case of the continuous culture with addition in a fed-batch manner, magnesium carbonate was produced at a concentration of 3 to 10 g/L and sodium hydrogen carbonate was produced at a concentration of 5 to 35 g/L in the culture medium, and then added at a velocity of 3 ml/hr.

Further, the culture was carried out at a pH of the culture fluid in a range of 6 to 7 and stabilized at a temperature between 35° C. and 40° C., and then, bubbles were removed with a foam breaker and a silicon antifoamer.

6. Operation of Continuous Culture

The continuous culture according to the present invention was carried out by using the above-described culture medium and varying a flow velocity of the culture medium between 0 ml/hr and 100 ml/hr. In the case of a flow velocity of 0 ml/hr, batch culture without supply or discharge of the medium was carried out.

Further, as an example, in the case of culture using a cell recycled system configured to recycle microbial cells, a flow velocity of a pump for recycle was adjusted to be ½ of a flow velocity of the sterilized culture medium introduced into the bioreactor.

Carbon dioxide was aerated at a speed of 0.6 vvm (aeration volume/medium volume/minute), and the culture was carried out while constantly maintaining hydrogen ions with sodium hydroxide. In order to supply carbonate ions such as magnesium carbonate and/or sodium hydrogen carbonate, a solution in which magnesium carbonate was concentrated at a concentration of 3 to 10 g/L and sodium hydrogen carbonate was concentrated at a concentration of 5 to 40 g/L was used and introduced into the bioreactor at a flow velocity of 3 ml/hr by using a peristaltic pump. Further, as an example of the present invention, there was provided a recycled system configured to recycle carbon dioxide discharged from the bioreactor and supply the carbon dioxide into the bioreactor again.

The reason why the material such as magnesium carbonate or sodium hydrogen carbonate that is dissolved in water and forms carbonate ions was added to the concentrate is that sufficient supply of carbon dioxide is essential for the Actinobacillus succinogenes as the producing strain to biosynthesize succinic acid. Further, bacteria use a pump protein present in the cell membrane to actively and readily transport the carbonate ions dissolved in water to use the carbon dioxide. Therefore, according to this logic, a process of directly adding carbonate ions to the bioreactor was further included to increase productivity of succinic acid.

In order to supply carbonate ions such as magnesium carbonate and/or sodium hydrogen carbonate, a solution in which magnesium carbonate was concentrated at a concentration of 10 to 40 g/L and sodium hydrogen carbonate was concentrated at a concentration of 5 to 40 g/L was used and introduced into the bioreactor at a flow velocity of 3 ml/hr by using a peristaltic pump.

As an example of the present invention, in the case of the fed-batch type continuous culture, a strain was inoculated into a culture fluid including 50 g/l of glucose, 5 g/l of yeast extract, 10 g/l of corn steep liquor, and 10 g/L of sodium hydrogen carbonate in an initial bioreactor, and 50 g/l of glucose, 5 g/l of yeast extract, and 10 g/l of corn steep liquor were supplied to a medium for continuous supply, and magnesium carbonate and sodium hydrogen carbonate were supplied in a fed-batch manner in addition to the medium.

Herein, the medium for continuous supply was supplied at a velocity of 1 to 100 ml/hr, the sodium hydrogen carbonate (NaHCO₃) was supplied at a concentration of 8 to 10 g/L at a velocity of 2 to 4 m/hr, and magnesium carbonate (MgCO₃) was supplied at a concentration of 10 to 30 g/L at a velocity of 2 to 4 m/hr.

7. Cell Recycling System

Actinobacillus succinogenes was inoculated into an initial culture medium including 50 μl of glucose, 5 g/l of yeast extract, 10 g/l of corn steep liquor, 20 g/L of magnesium carbonate, and 10 g/L of sodium hydrogen carbonate in the bioreactor of the present invention, and a liquid culture medium for continuous culture of cell recycled continuous culture was produced as follows. The liquid culture medium included 50 g/l of glucose, 5 μl of yeast extract, 10 μl of corn steep liquor, 20 g/L of magnesium carbonate, and 10 g/L of sodium hydrogen carbonate. The liquid culture medium for continuous culture was supplied at a velocity of 30 to 55 ml/hr at the same time when the culture fluid for culturing the Actinobacillus succinogenes was removed at a velocity of 30 to 55 ml/hr.

Further, in order to maintain a high concentration of microbial cells as an example, the present inventors added a cell recycled process in which the Actinobacillus succinogenes as the producing strain was collected by filtering from the removed culture, and washed and collected, and a culture fluid containing the collected Actinobacillus succinogenes was supplied at a velocity of 15 to 22.5 ml/hr. Thus, the continuous culture was carried out at a high concentration of the microbial cells for producing succinic acid.

### EXAMPLE 2

Continuous Culture Process with Drip Tube

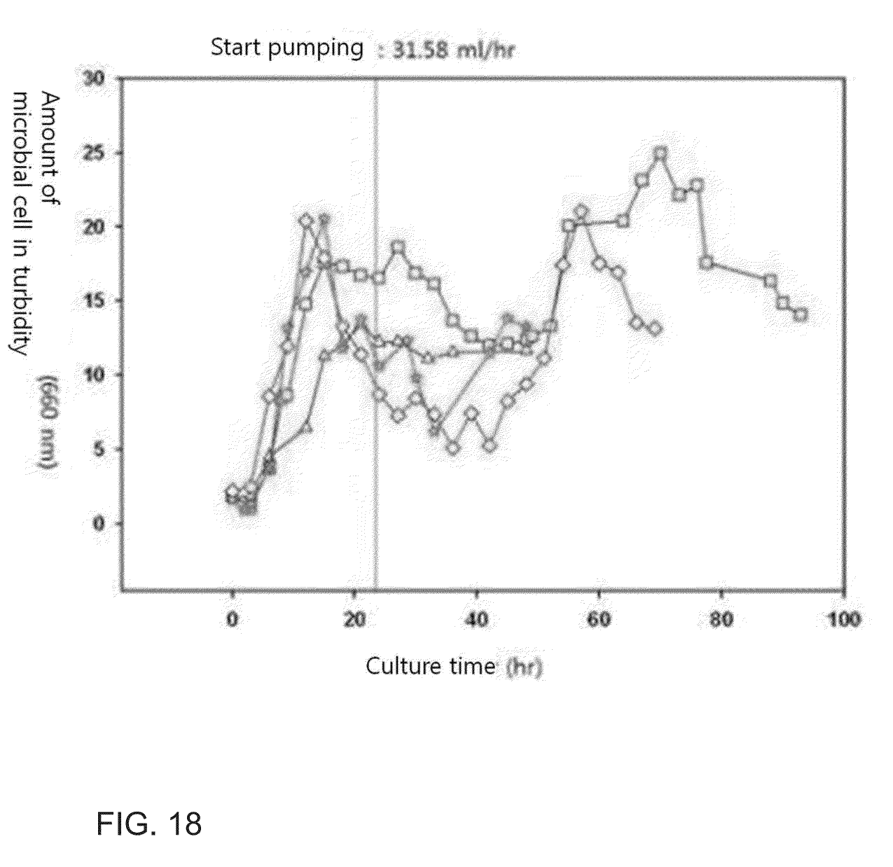

The conventional cell recycled process using Actinobacillus succinogenes has a problem that a microorganism having a high cell specific growth velocity of microbial cells often contaminates a pure culture medium tank through an inlet for supplying a pure culture medium (medium for continuous culture) to a bioreactor or a culture tank. As shown in FIG. 18, when a pure culture medium is contaminated with Actinobacillus succinogenes during continuous culture, an amount of microbial cells is sharply increased as can be seen in the case of cell recycled culture. Such a phenomenon occurs since a culture fluid in the bioreactor has a little viscosity and aerated carbon dioxide adheres to the culture medium inlet in the bioreactor. It was confirmed that in most cases where bubbles were formed during culture, such a phenomenon occurred.

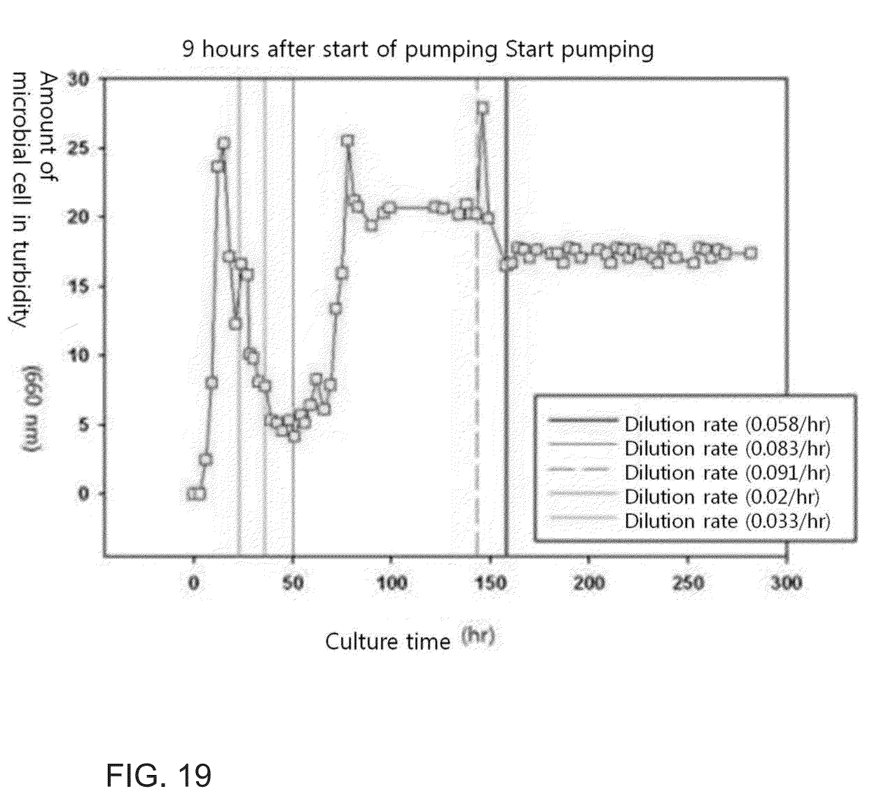

Further, FIG. 19 is a graph showing a change in microbial cells in continuous culture after a drip tube is provided. It was observed that the operation was smoothly carried out for about 300 hours.

1. Manufacturing of Drip Tube

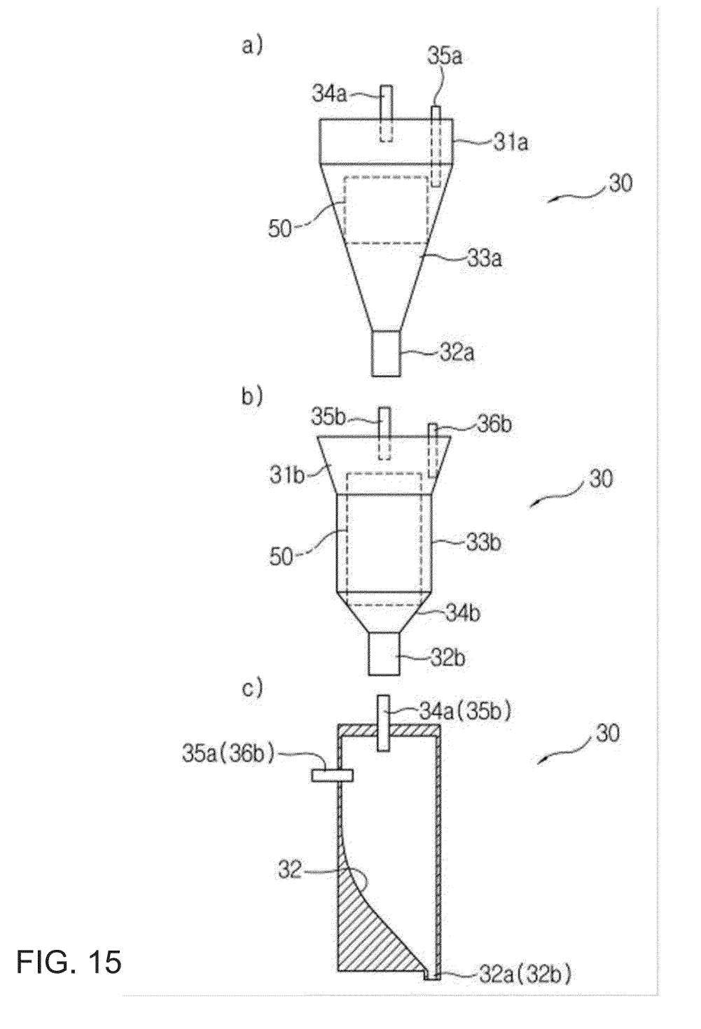

The present inventors manufactured two types of drip tubes as depicted in FIG. 15.

A. Carbon Dioxide Pressurization Type Drip Tube

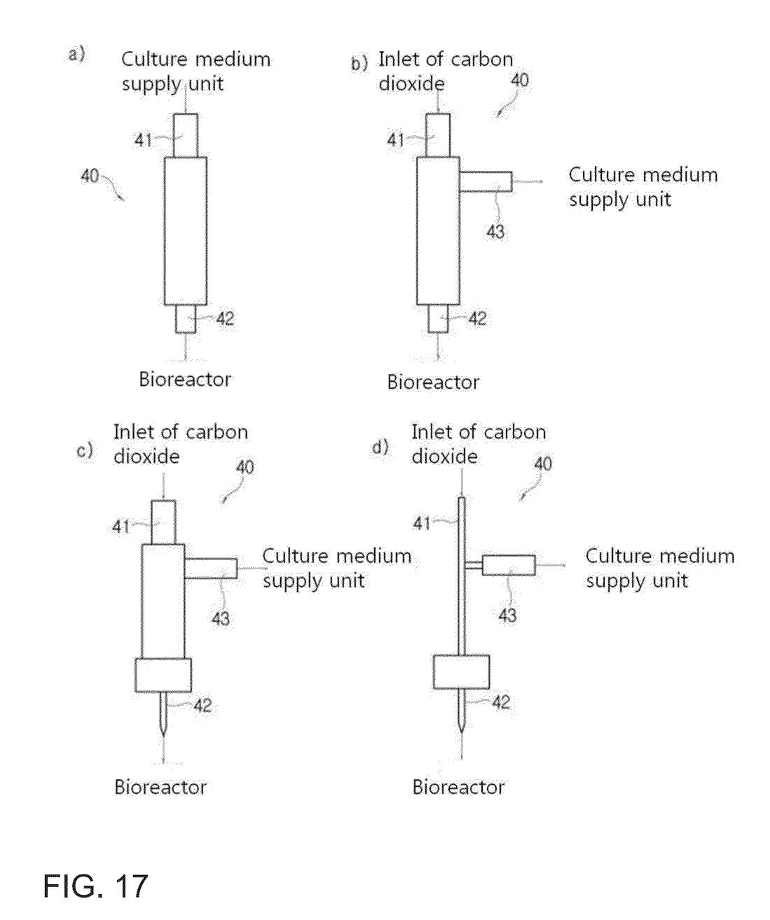

A carbon dioxide pressurization type drip tube as shown on the left of FIG. 17 (FIG. 17(a)) is characterized in that aerated carbon dioxide is recycled and a culture medium inlet is pressurized by the gas, and it is very effective for a dilution rate of a low flow velocity. According to a result of the experiment, it was very effective for a flow velocity of about 0 to about 50 ml/hr, and in the case of a flow velocity higher than 50 ml/hr, it was often observed that a fluid pressure of the culture medium was increased and the culture medium flowed to a line pressurized by the gas.

B. Gravity Type Drip Tube

In a gravity type drip tube as shown on the right of FIG. 17 (FIG. 17(b)), when microbial cells adhering to an inlet and grown along a culture medium due to a density difference caused by gravity have a density higher than that of the culture medium, they climb down again. As a height is increased, the effect is increased. According to a result of the experiment in the present invention, as shown on the right of FIG. 17, it was observed that the operation was carried out very well even at a height of about 16 cm.

The pump was operated at various dilution rates (l/hr) by using the manufactured drip tube, and as a result of culture of succinic acid, as shown in FIG. 19, the culture was smoothly carried for about 300 hours when as compared with the conventional continuous culture for 60 to 80 hours in the same conditions. Even in the test for longer than 300 hours, Actinobacillus succinogenes did not return to and grow in the pure culture medium tank.

### EXAMPLE 3

1. Continuous Culture Process with Cell Separator

Cell recycled continuous culture is characterized in that due to a great amount of microbial cells, culture can be carried out at a dilution rate of a higher velocity than a cell specific growth velocity of a producing strain in the conventional continuous culture and can produce microbial cells at a high concentration with high productivity as compared with the conventional continuous culture. In a continuous fermentation process of bacteria, most of the conventional cell separators used for a cell recycled process use a membrane or a filter. However, when a membrane or a filter is used for cell separation, various factors such as a size of a microbial cell, viscosity of a culture fluid, etc. may cause problems. Such problems result in clogging of the membrane or the filter. Such a problem reduces efficiency of cell recycle and makes it difficult to smoothly proceed with continuous culture.

In order to solve this problem, the present inventors intended to design a cell separator capable of effectively separating cells, and manufactured a cell separator as depicted in FIG. 15. The cell separator of the present invention separates microbial cells by using a density difference caused by gravity according to the same principle as that of the above-described drip tube and includes a separation tube therein to increase efficiency.

By using the manufactured cell separator, cell recycled culture was carried out. A dilution rate was about 40 ml/hr, and a recycle rate was about 20 ml/hr which was about ½ of the dilution rate. Cell recycled continuous culture was carried out with a culture medium including 54.5 g/L of glucose, 6.5 g/L of yeast extract, 9.5 g/L of corn steep liquor solids, 10 g/L of sodium hydrogen carbonate, and 20 g/L of magnesium carbonate.

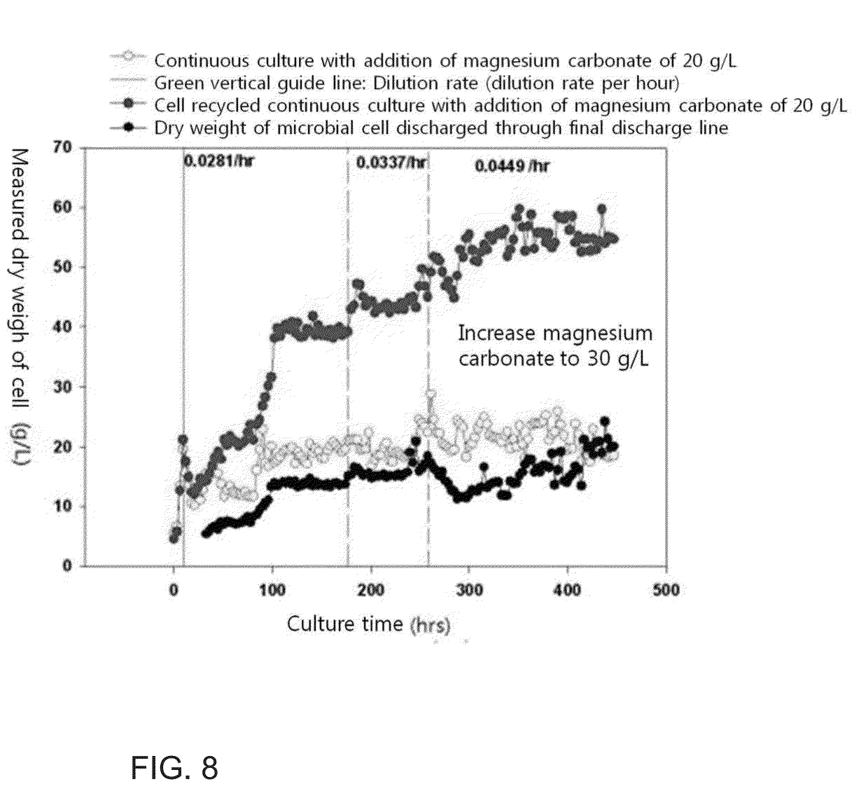

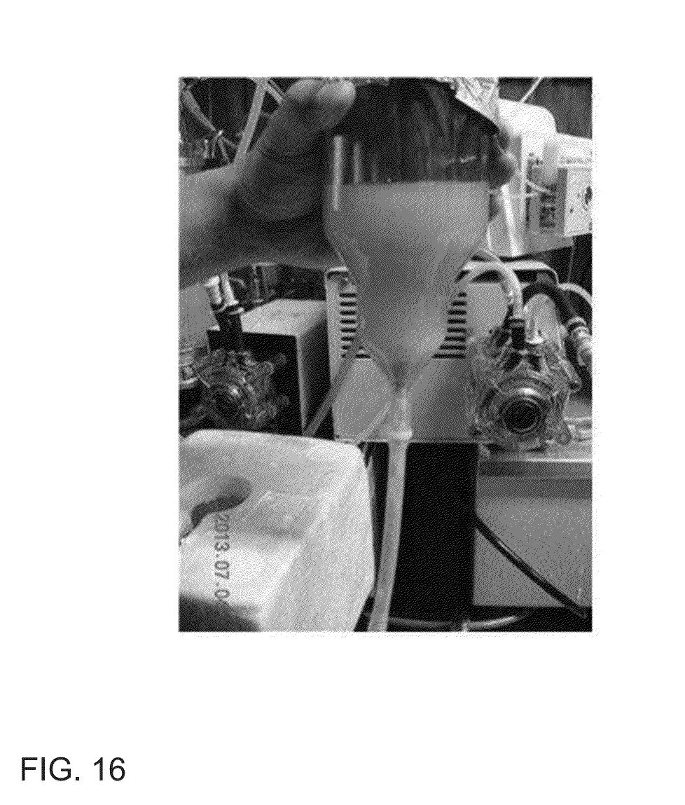

According to a result of the experiment, as shown in FIG. 16, the microbial cells and the culture fluid were separated due to a density difference, and sedimentation on a bottom portion of the cell separator was observed. As shown in FIG. 8, it could be seen that an amount of microbial cells in the cell recycled continuous culture was increased about 3 times as compared with an amount of microbial cells in the continuous culture, and as a result of analysis on an amount of microbial cells in a final discharge line at a ratio of a pure culture medium supply rate to a recycle rate of 1:0.5, the cell separator collected about 65% or more of the microbial cells from the culture fluid discharged.

### EXAMPLE 4

Connection Structure Between Drip Tube and Cell Separator

A microbial cell reuse system 10 of the present invention includes a bioreactor 20 in which a culture fluid is stored, a cell separator 30 configured to separate the culture fluid supplied to the bioreactor 20 into microbial cells and the culture fluid through a separation means 50 and discharge them, a drip tube 40 configured to supply a culture medium to the bioreactor 20, and a collection line 60 configured to collect the microbial cells discharged from the cell separator 30 and supply the microbial cells to the bioreactor 20.

The bioreactor 20 has a certain size and includes an accommodation space 21 that accommodates the culture fluid therein. At an upper part of the bioreactor 20, there is formed a bioreactor inlet 22 through which the culture medium and the microbial cells are supplied, and at a lower part thereof, there is formed a bioreactor outlet 23 through which the culture fluid accommodated in the bioreactor 20 is discharged.

That is, in the bioreactor 20, the culture medium supplied through the drip tube 40 is supplied to the accommodation space 21 through the bioreactor inlet 22 and then the culture fluid containing the culture medium and microbial cells and accommodated in the accommodation space 21 is transferred to the cell separator 30 through the bioreactor outlet 23.

The cell separator 30 configured to separate and discharge the culture fluid supplied from the bioreactor 20 separates the culture fluid supplied from the bioreactor 20 into the microbial cells and the culture fluid through the separation means 50. Herein, for smooth transfer of the culture fluid or the microbial cells, a pump may be provided between the bioreactor 20 and the cell separator 30, but explanation thereof will be omitted.

Herein, the separation means 50 may be optionally employed from publicly-known means capable of separating a culture fluid and microbial cells. In the present invention, one of a cylindrical tube capable of separating microbial cells and a culture fluid by using specific gravity and a filter comprised of multiple mesh net layers may be optionally included.

Further, in the present invention, the cell separator 30 has any one of structures as shown in FIGS. 15(a) to 15(c). Detailed explanation thereof will be provided below.

The cell separator 30 shown in FIG. 15(a) has a certain size and includes an upper vertical portion 31a formed at its upper part and a culture fluid outlet 32a having a smaller diameter than the upper vertical portion and provided under the upper vertical portion 31a with a space between them, and an inclined portion 33a is formed between the upper vertical portion 31a and the culture fluid outlet 32a.

That is, the cell separator 30 shown in FIG. 15(a) is configured to rapidly transfer the culture fluid separated through the separation means 50 to the culture fluid outlet 32a by using the inclined portion 33a formed between the upper vertical portion 31a and the culture fluid outlet 32a.

Herein, at an upper part of the upper vertical portion 31a, a culture fluid supply unit 34a configured to be supplied with the culture fluid from the bioreactor 20 and a tube connection portion 35a configured to supply the microbial cells separated through the separation means 50 to the collection line 60 are formed.

Further, the cell separator 30 shown in FIG. 15(b) has a certain size and includes an upper inclined portion 31b formed at an upper part, a culture fluid outlet 32b having a smaller diameter than the upper inclined portion 31b and provided under the upper inclined portion 31b with a space between them, and an upper vertical connection portion 33b and a lower inclined connection portion 34b are continuously formed between the upper inclined portion 31b and the culture fluid outlet 32b.

That is, the cell separator 30 shown in FIG. 15(b) is configured to accommodate a large amount of the culture fluid and separate the culture fluid and the microbial cells through the separation means 50 by using the upper vertical connection portion 33b and the lower inclined connection portion 34b formed between the upper inclined portion 31b and the culture fluid outlet 32b. Herein, at an upper part of the upper inclined portion 31b, a culture fluid supply unit 35b configured to be supplied with the culture fluid from the bioreactor 20 and a tube connection portion 36b configured to supply the microbial cells separated through the separation means 50 to the collection line 60 are formed.

Furthermore, the cell separator 30 shown in FIG. 15(c) includes a curved surface guiding portion 32 that allows the culture fluid to be rapidly discharged through the culture fluid outlets 32a and 32b. Herein, the curved surface guiding portion 32 may be formed in a curved surface or a combination of a curved surface or an inclined surface.

The drip tube 40 configured to supply the culture medium to the bioreactor 20 supplies the culture medium supplied from a culture medium supply unit 70 to the bioreactor 20 with regulation of a flow velocity. Further, in the present invention, the drip tube 40 has any one of structures as shown in FIGS. 17(a) and 17(b).

The drip tube 40 shown in FIG. 17(a) has a certain size and includes a tube connection portion 41 connected to the culture medium supply unit 70 configured to supply the culture medium at its one side and a velocity control portion 42 configured to control a flow velocity of the supplied culture medium at a portion connected to the bioreactor 20.

That is, the drip tube 40 is configured to supply the culture medium to the bioreactor 20 while the velocity control portion 42 controls a flow velocity of the culture medium supplied through the tube connection portion 41. Herein, the velocity control portion 42 configured to control a flow velocity of the culture medium controls a supply rate of the culture medium by using a change in diameter of a cross section or a publicly-known fluid control mechanism, but explanation thereof will be omitted.

Further, the drip tube 40 shown in FIG. 17(b) has a certain size and includes a tube connection portion 43 connected to the culture medium supply unit 70 configured to supply the culture medium at its one side, the velocity control portion 42 configured to control a flow velocity of the supplied culture medium at a portion connected to the bioreactor 20, and a carbon dioxide inlet 41 configured to recycle carbon dioxide accommodated in the drip tube 40 or to introduce the carbon dioxide to a preset position and formed at its one side.

That is, the drip tube 40 includes the carbon dioxide inlet 43 at its one side to prevent the culture medium from being contaminated with the carbon dioxide accommodated in the drip tube 40 at the same time when the culture medium is supplied to the bioreactor 20 while the velocity control portion 42 controls a flow velocity of the culture medium supplied through the tube connection portion 41.

The collection line 60 configured to collect the microbial cells discharged from the cell separator 30 and supply the microbial cells to the bioreactor 20 supplies the bioreactor 20 with the culture medium discharged to the outside through the separation means 50 of the cell separator 30. To do so, the collection line 60 includes a hose 61 having a certain length and a pump 62 provided in the middle of the hose 61 and configured to transfer the culture medium to the bioreactor 20.

### EXPERIMENTAL EXAMPLE 1

1. Quantitative Analysis on Succinic Acid

The present inventors collected a sample into a 1.4 ml micro tube for quantitative analysis on succinic acid as a target product of the present invention from a culture fluid in which Actinobacillus succinogenes was cultured, diluted the sample by serial dilution, and carried out a filtering process twice with a 0.45 μm filter paper. Then, an analysis was carried out by using HPLC in the following conditions.

Analysis temperature: 25° C.

Flow velocity: 0.8 ml/min

Mobile phase: 0.01N H₂SO₄

Analysis time: 20 minutes

Sample injection amount: 10 μl

Column: Organic acid column (Bio-Rad, Aminex HPX 87H, 125-0140)

Detector: UV detector

Detection wavelength: 210 nm

2. Carbohydrate Analysis

For carbohydrate analysis on the culture fluid, the culture fluid was centrifuged at 12,000 rpm for 10 minutes and a supernatant was taken out, and then the culture fluid was centrifuged repeatedly three times at 12,000 rpm for 10 minutes and only a supernatant was taken out and filtered with a 0.45 μm filter paper for HPLC. The carbohydrate analysis was carried out by using HPLC in the following conditions.

Analysis temperature: 40° C.

Flow velocity: 1.2 ml/min

Mobile phase: acetonitrile:water=75:25 (v/v)

Analysis time: 15 minutes

Sample injection amount: 20 μl

Column: Amine column (250 mm×46 mm, RS tech)

Detector: RI detector

3. Check of Amount of Microbial Cells of Actinobacillus Succinogenes

The culture fluid cultured in the bioreactor was collected and centrifuged at 12,000 rpm for 10 minutes and washed three or more times with distilled water or saline water. Then, it was dried at 100° C. for 10 to 12 hours and its weight was measured. Otherwise, the collected culture fluid was uniformly mixed, and absorbance was measured by using a spectrophotometer at 660 nm. An amount of microbial cells can be measured in turbidity. If a production medium containing magnesium carbonate was cultured, a crude liquid was diluted 50 times by using 1 N hydrochloric acid to completely dissolve the magnesium carbonate and then absorbance was measured.

4. Calculation Formulas of Culture Variables for Amount of Microbial Cells, Production Amount of Succinic Acid, and Glucose Used

Formulas applied to a continuous culture process and cell recycled fermentation process are as provided below, and the terms used in the following formulas are as follows.

V: Bioreactor volume, F: Flow velocity, D: Dilution rate, X: Amount of microbial cells in bioreactor, P: Production amount of succinic acid in bioreactor, S0: Initial glucose concentration, S: Glucose concentration in bioreactor, γX: Microbial cell growth velocity per hour per volume, γS: Consumption velocity of glucose per hour per volume, and γP: Production velocity of succinic acid per hour per volume

A. In the case of continuous culture, a microbial cell resin was calculated as follows.

$${FX}_{0} - {FX}_{1} + {V}_{{γ}_{{X}_{1}}} = V \frac{d {X}_{1}}{d t}$$

$$steady state , \frac{d {X}_{1}}{d t} = 0$$

$${γ}_{X} = DX$$

A substrate resin was calculated as follows.

$${FS}_{0} - {FS}_{1} - V \frac{μ}{{Y}_{X / S}} {X}_{1} = V \frac{d {S}_{1}}{d t}$$

$$steady state , \frac{d {S}_{1}}{d t} = 0$$

$${γ}_{S} = D ( {S}_{0} - S )$$

A product resin was calculated as follows.

$${FP}_{0} - {FP}_{1} + {V}_{{γ}_{{P}_{1}}} = V \frac{d {P}_{1}}{d t}$$

$$steady state , \frac{d {P}_{1}}{d t} = 0$$

$${γ}_{P} = DP$$

A microbial cell with respect to glucose, a production amount of succinic acid with respect to glucose, and a production amount of succinic acid with respect to a microbial cell satisfying the above formulas were defined and analyzed as follows by using the above illustration.

$${Y}_{X / S} = \frac{{γ}_{X}}{{γ}_{S}} {Y}_{P / S} = \frac{{γ}_{P}}{{γ}_{S}} {Y}_{P / X} = \frac{{γ}_{P}}{γ X }$$

B. In the case of a cell recycled continuous culture process, a microbial cell resin was calculated as follows.

$$\frac{F}{V} {X}_{0} + \frac{α F}{V} {CX}_{1} - \frac{( 1 + α )}{V} {FX}_{1} + {γ}_{{X}_{1}} = \frac{d {X}_{1}}{d t}$$

$$steady state , \frac{d {X}_{1}}{d t} = 0$$

$${γ}_{X} = D ( 1 + α ) X - D α CX$$

A substrate resin was calculated as follows.

$$\frac{F}{V} {S}_{0} + \frac{α F}{V} {S}_{1} - \frac{( 1 + α )}{V} {FS}_{1} - \frac{μ}{{Y}_{X / S}} {X}_{1} ( {γ}_{{S}_{1}} ) = \frac{d {S}_{1}}{d t}$$

$$steady state , \frac{d {S}_{1}}{d t} = 0$$

$${γ}_{S} = {DS}_{0} + D α S - ( 1 + α ) DS$$

$${γ}_{P} = ( 1 + α ) P$$

A microbial cell with respect to glucose, a production amount of succinic acid with respect to glucose, and a production amount of succinic acid with respect to a microbial cell satisfying the above formulas were defined and analyzed as follows by using the above illustration.

$${Y}_{X / S} = \frac{{γ}_{X}}{{γ}_{S}} {Y}_{P / S} = \frac{{γ}_{P}}{{γ}_{S}} {Y}_{P / X} = \frac{{γ}_{P}}{γ X }$$

### EXPERIMENTAL EXAMPLE 2

Comparison Between Continuous Culture Added with Magnesium Carbonate and Continuous Culture

The present inventors carried out the following experiment in order to compare productivity of continuous culture depending on addition of magnesium carbonate according to the above-described Examples.

A production medium without containing magnesium carbonate was diluted at an increasing dilution rate from 12 ml/hr to 24 ml/hr in a bioreactor having a volume of 1.2 L. Further, magnesium carbonate was added in a fed-batch manner at a flow velocity of 3 ml/hr.

1. Amount of Microbial Cells and Glucose Consumption Rate

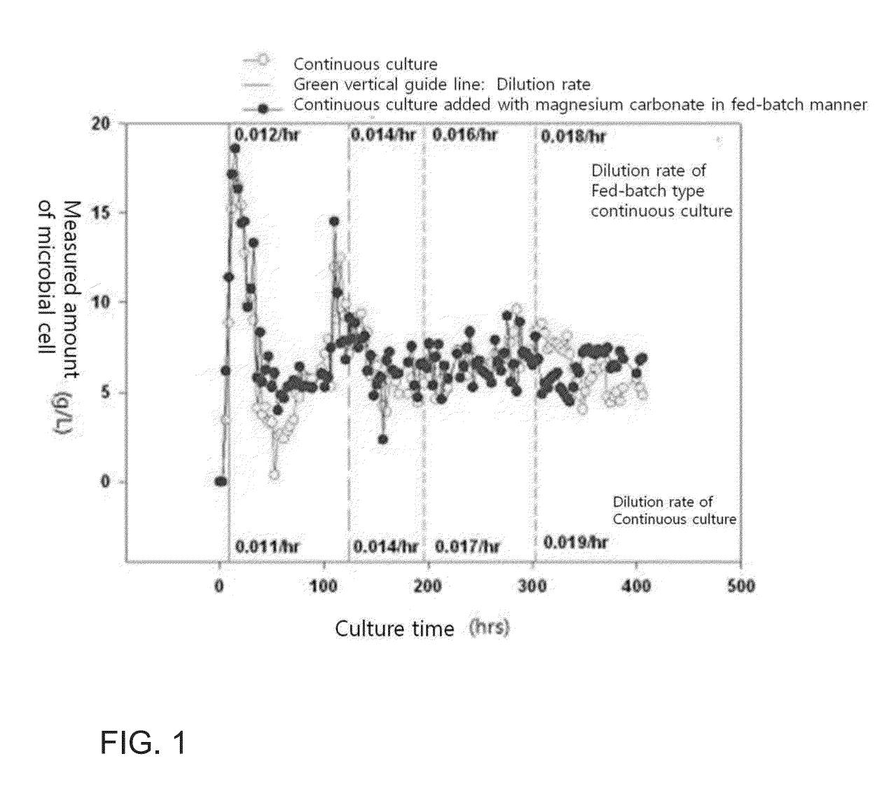

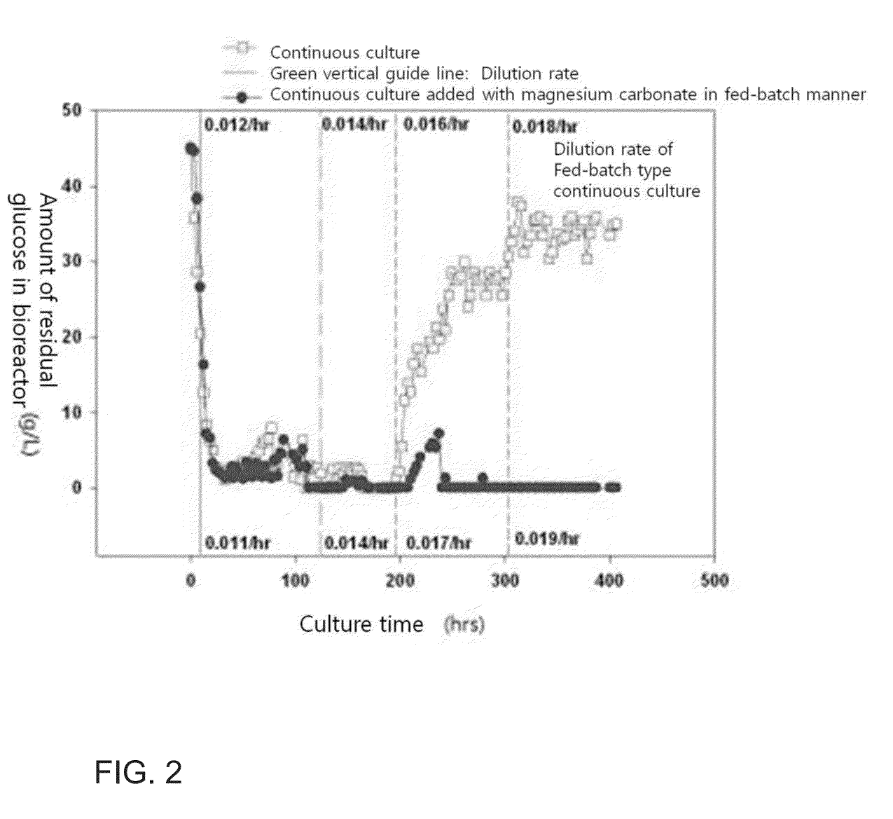

According to an analysis result, as shown in FIG. 1, there was no big difference in amount of microbial cells. However, as shown in FIG. 2, there was a clear difference in amount of residual glucose.

Further, it was observed that unlike the continuous culture in which as a dilution rate was increased, an amount of residual glucose was sharply increased, the continuous culture process added with carbonate ions still consumed the whole amount of glucose.

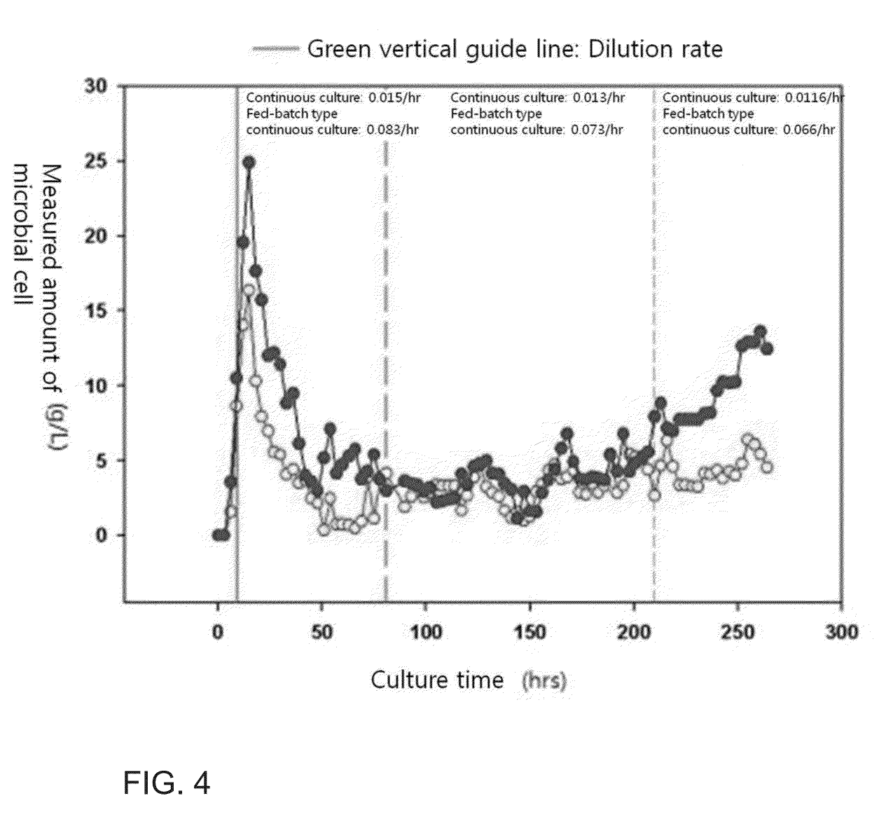

Therefore, based on this result, it was concluded that a dilution rate which could not be overcome in the continuous culture could be overcome in the case of addition of carbonate ions, and it could be seen that a production amount of succinic acid was increased (refer to FIG. 4).

Further, when the added magnesium carbonate was introduced into the bioreactor, it was diluted in the total volume and thus its concentration was very small. However, it could be seen that even when a very small amount of magnesium carbonate was added, an excellent effect of using glucose could be shown.

2. Productivity of Succinic Acid

According to the analysis results as shown in Table 1, it could be seen that productivity of succinic acid in the batch culture was about 0.339 g per hour per liter, and productivity in the continuous culture was increased at a dilution rate of 0.015 or more as compared with the productivity in the batch culture. Further, the continuous culture with addition of magnesium carbonate produced high productivity of 0.469 g per hour per liter at a low dilution rate (l/hr) of 0.012 as compared with the batch culture, and also produced high productivity of 0.481 g even at a final dilution rate of 0.018 as compared with the batch culture (a value obtained by dividing a final production amount of succinic acid by the total culture time in the batch culture, and a value obtained by multiplying DP, a dilution rate, and a production amount of succinic acid for a corresponding time in the continuous culture).

Furthermore, since an effect of magnesium carbonate was confirmed, it could be confirmed that when magnesium carbonate was used at a concentration of about 20 g/L from the beginning and cultured without addition in a fed-batch manner, the whole amount of glucose was consumed up to 0.0449 and then a dilution rate could be further increased (refer to Table 1). Moreover, it could be confirmed that productivity of succinic acid was 0.671 g per hour per liter at a dilution rate of 0.0449 l/hr. An amount of microbial cells in the culture medium containing magnesium carbonate was also increased by 2 times to nearly 4 times as compared with the case without containing magnesium carbonate (refer to Table 1).

TABLE 1
Comparison in Productivity of Continuous Culture Depending on Supply of MgCO₃

| | Batch | Continuous Culture | | | | | | | | | |
|---|---|---|---|---|---|---|---|---|---|---|---|
| Bioreactor | Culture | Non-addition of MgCO₃ | | | | | | | Addition of MgCO₃ | | |
| Dilution rate | — | 0.011 | 0.0116 | 0.013 | 0.014 | 0.015 | 0.017 | 0.019 | 0.281 | 0.0337 | 0.0449 |
| Steady X | 7.85 | 4.396 | 7.810 | 3.813 | 5.641 | 4.486 | 6.721 | 5.303 | 19.078 | 19.885 | 21.632 |
| Steady S | 0 | 6.464 | 5.280 | 4.990 | 1.079 | 7.245 | 27.634 | 33.987 | 0.284 | 0.000 | 0.000 |
| Steady P | 29.47 | 25.447 | 27.681 | 20.677 | 23.727 | 23.595 | 22.948 | 17.378 | 14.650 | 14.632 | 14.924 |
| qs | 0.119 | 0.096 | 0.059 | 0.136 | 0.109 | 0.126 | 0.044 | 0.039 | 0.659 | 0.076 | 0.093 |
| qp | 0.078 | 0.064 | 0.041 | 0.070 | 0.059 | 0.079 | 0.058 | 0.062 | 0.022 | 0.026 | 0.031 |
| γX | 0.164 | 0.048 | 0.091 | 0.050 | 0.079 | 0.067 | 0.114 | 0.101 | 0.536 | 0.719 | 0.972 |
| γS | 0.938 | 0.424 | 0.461 | 0.520 | 0.615 | 0.566 | 0.295 | 0.209 | 1.257 | 1.614 | 2.022 |
| DP | 0.61 | 0.280 | 0.321 | 0.269 | 0.332 | 0.354 | 0.390 | 0.330 | 0.412 | 0.523 | 0.671 |
| Y_{X/S} | 0.174 | 0.114 | 0.197 | 0.095 | 0.128 | 0.119 | 0.387 | 0.482 | 0.427 | 0.442 | 0.481 |
| Y_{P/X} | 3.754 | 5.789 | 3.544 | 5.422 | 4.206 | 5.259 | 3.414 | 3.277 | 0.328 | 0.325 | 0.332 |

Dilution rate: Dilution rate D = F/V; hU
Recycled continuous: Cell recycled continuous culture
Steady X: Amount of microbial cells in a steady state in a bioreactor (g/L)
Steady S: Amount of residual glucose in a steady state in a bioreactor (g/L)
Steady P: Production amount of succinic acid in a steady state in a bioreactor (g/L)
qs: Consumption rate of glucose with respect to cell (glucose (g)/cell (g)/hour)
qp: Production rate of succinic acid with respect to cell (succinic acid (g)/cell (g)/hour)
γX: Microbial cell growth velocity (amount of microbial cell (g)/liter/hour)
γS: Consumption rate of glucose (glucose (g)/liter/hour)
DP: Production velocity of succinic acid (succinic acid (g)/liter/hour)
Y_{X/S}: Yield rate of microbial cells with respect to glucose (microbial cell (g)/glucose (g))
Y_{P/S}: Yield rate of succinic acid with respect to glucose (succinic acid (g)/glucose (g))
Y_{P/X}: Yield rate of succinic acid with respect to amount of microbial cells (succinic acid (g)/microbial cell (g))

### EXPERIMENTAL EXAMPLE 3

Continuous Culture Depending on Addition of Sodium Hydrogen Carbonate (NaHCO₃)

The present inventors carried out the following experiment in order to check an effect of addition of sodium hydrogen carbonate after confirming that the effect of the continuous culture caused by addition of magnesium carbonate was further increased in the above-described Example.

Like magnesium carbonate, sodium hydrogen carbonate is a representative material for supplying carbonate ions. By using a high solubility of sodium hydrogen carbonate, sodium hydrogen carbonate was concentrated at a concentration of about 30 g/L and magnesium carbonate was concentrated at a concentration of about 5 g/L equal to the previous case to produce a concentrate, and the experiment was carried out. The concentrate was added at a flow velocity of 3 ml/hr, and the other microorganism culturing conditions or bioreactor operation conditions were the same as described in Example 1.

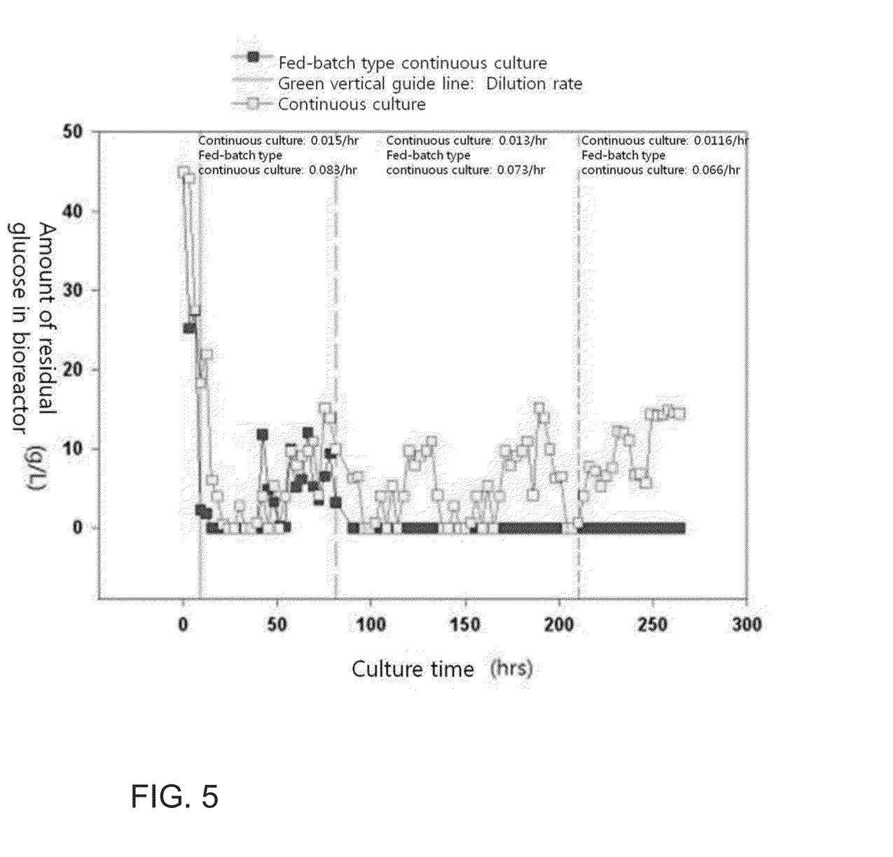

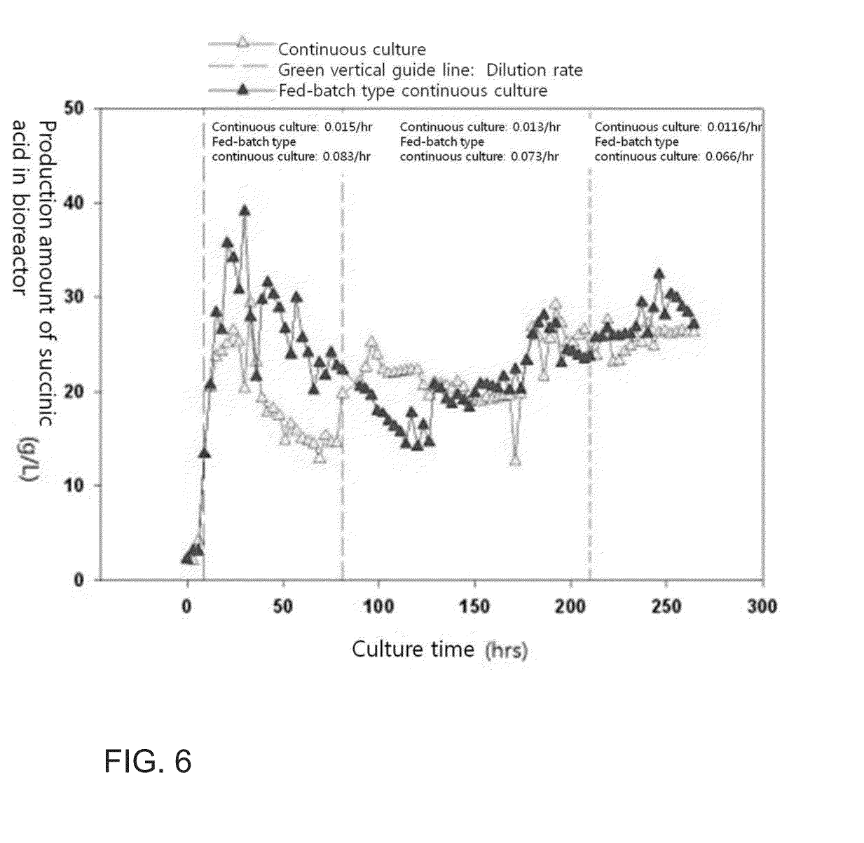

According to the analysis results, it was observed that when the two materials were added together, an effect was remarkably high as compared with the case where only magnesium carbonate was added. According to the result shown in FIG. 4, a cellular increment was observed marginally, but as shown in FIG. 5, as compared with the continuous culture in which glucose was not fully used, the whole amount of glucose was used in the continuous culture with addition of carbonate ions. Further, as shown in FIG. 6, it was observed that succinic acid was slightly increased, but there was a difference of 6 to 8 times in dilution rate between the two experiments.

Therefore, it can be seen that even at a dilution rate 6 to 8 times higher, a result of the continuous culture with addition of carbonate ions tended to be similar to or slightly higher than a result of the continuous culture.

Further, according to Table 2, it can be seen that in the continuous culture with addition of magnesium carbonate and sodium hydrogen carbonate, almost the whole amount of glucose was used even at a dilution rate 6 times or higher and productivity of succinic acid was increased by about 3 times up to 1.390 g/L/hr as compared with the continuous culture with addition of magnesium carbonate. Such a value was 4 time or higher than the batch culture. A 4-times increase in productivity means that when succinic acid is produced by continuous culture added in a fed-batch manner, even if a production scale of a bioreactor is decreased by 4 times, products in the same amount can be produced. Therefore, it is expected to be more competitive for industrialization of succinic acid.

TABLE 2
Comparison in Productivity of Continuous
Culture Depending on Supply of NaHCO₃

| | | Continuous Culture | | | | | | |
|---|---|---|---|---|---|---|---|---|
| | Batch | | | | | Addition of MgCO3 | | |
| Bioreactor | Culture | Addition of MgCO₃ | | | | and NaHCO₃ | | |
| Dilution rate | — | 0.012 | 0.014 | 0.016 | 0.018 | 0.066 | 0.073 | 0.083 |
| Steady X | 7.85 | 6.034 | 5.952 | 6.680 | 6.924 | 4.388 | 3.023 | 0.935 |
| Steady S | 0 | 2.879 | 0.226 | 1.045 | 0.000 | 0.000 | 0.000 | 0.000 |
| Steady P | 29.47 | 40.365 | 36.078 | 28.204 | 26.056 | 25.519 | 19.038 | 14.886 |
| qₛ | 0.119 | 0.062 | 0.086 | 0.090 | 0.104 | 0.651 | 1.050 | 3.876 |
| qₚ | 0.078 | 0.078 | 0.084 | 0.068 | 0.069 | 0.384 | 0.460 | 1.322 |
| Y_{X} | 0.164 | 0.070 | 0.083 | 0.108 | 0.128 | 0.290 | 0.221 | 0.078 |
| Y_{S} | 0.938 | 0.377 | 0.510 | 0.598 | 0.718 | 2.858 | 3.173 | 3.623 |
| DP | 0.61 | 0.469 | 0.501 | 0.456 | 0.481 | 1.684 | 1.390 | 1.236 |
| Y_{x/s} | 0.174 | 0.186 | 0.162 | 0.181 | 0.178 | 0.101 | 0.070 | 0.021 |
| Y_{P/X} | 3.754 | 6.689 | 6.062 | 4.222 | 3.763 | 5.815 | 6.298 | 15.927 |
| Yp/s | 0.655 | 1.244 | 0.984 | 0.762 | 0.670 | 0.589 | 0.438 | 0.341 |

### EXPERIMENTAL EXAMPLE 4

Comparison in Productivity of Continuous Culture Process Depending on Cell Recycled Process

The present inventors carried out the continuous culture without a cell recycled system and the continuous culture added with a cell recycled system in order to check an effect of a cell recycled continuous culture process on productivity of succinic acid according to the above-described Example, and compared an amount of microbial cells, an amount of glucose used, and productivity of succinic acid.

A culture medium was supplied at an increasing inflow velocity of from 33.7 ml/hr to 40.44 ml/hr and 53.88 ml/hr to a bioreactor having a volume of 1.2 L, and a recycle rate was about ½ of the inflow velocity of the sterilized culture medium.

1. Comparison in Amount of Microbial Cells

According to a result of analyzing an amount of microbial cells in the continuous culture process with or without the cell recycled system, as shown in FIG. 8 and Table 1, it could be seen that there was no big difference in amount of microbial cells between the continuous culture and the cell recycled process up until 30 hours after pumping, but thereafter, there was a clear difference and an amount of microbial cells in the cell recycled process was considerably increased.

In the case of the continuous culture, there was no big increase in amount of microbial cells which was 19.078 g/L at a dilution rate of 0.0281 l/hr, 19.885 g/L at a dilution rate of 0.0337 l/hr, and 21.632 g/L at a dilution rate of 0.0449 l/hr.

In the case of the cell recycled process, there was an increase in amount of microbial cells which was 39.325 g/L at a dilution rate of 0.0281 l/hr, 43.328 g/L at a dilution rate of 0.0337 l/hr, and 53.806 g/L at a dilution rate of 0.0449 l/hr.

Therefore, according to the experiment results, it could be seen that the amount of microbial cells in the cell recycled process was increased by about 2 times overall as compared with the continuous culture, and about 65% or more of the microbial cells discharged through a final discharge line were collected again in the bioreactor.

2. Comparison in Consumption Amount of Glucose

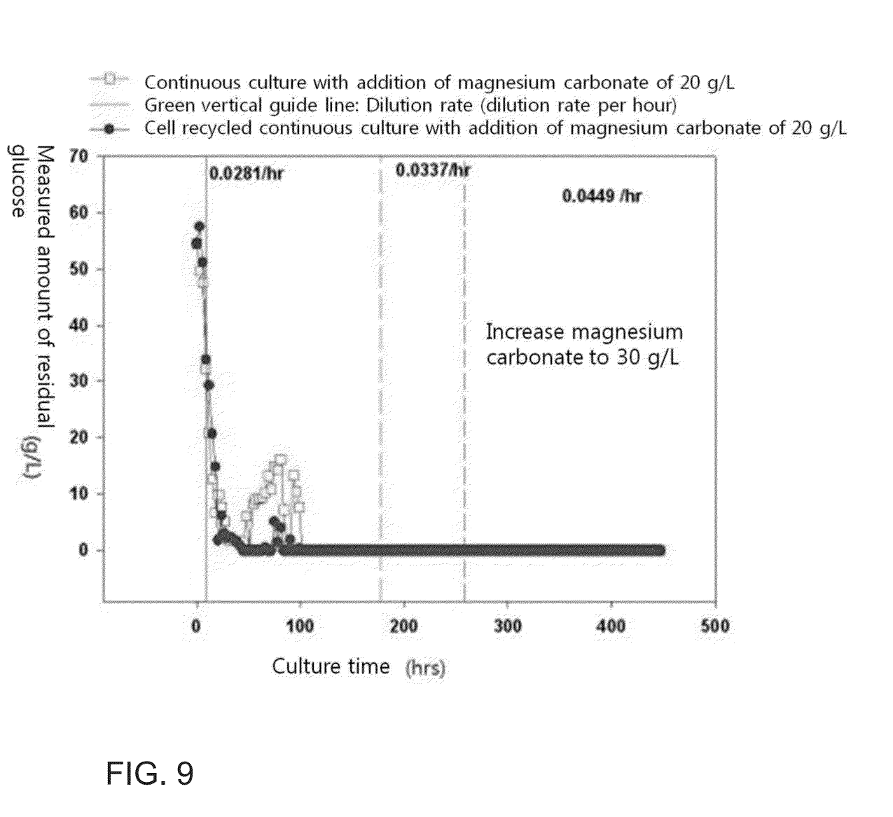

According to a result of analyzing an amount of glucose used in the continuous culture process with or without the cell recycled system, it could be seen that the whole amount of glucose was used in both the continuous culture and the cell recycled process. Based on this result, it could be confirmed that a dilution rate could be further increased in the both processes and could be still further increased in the cell recycled process (refer to FIG. 9).

3. Comparison in Production Amount of Succinic Acid

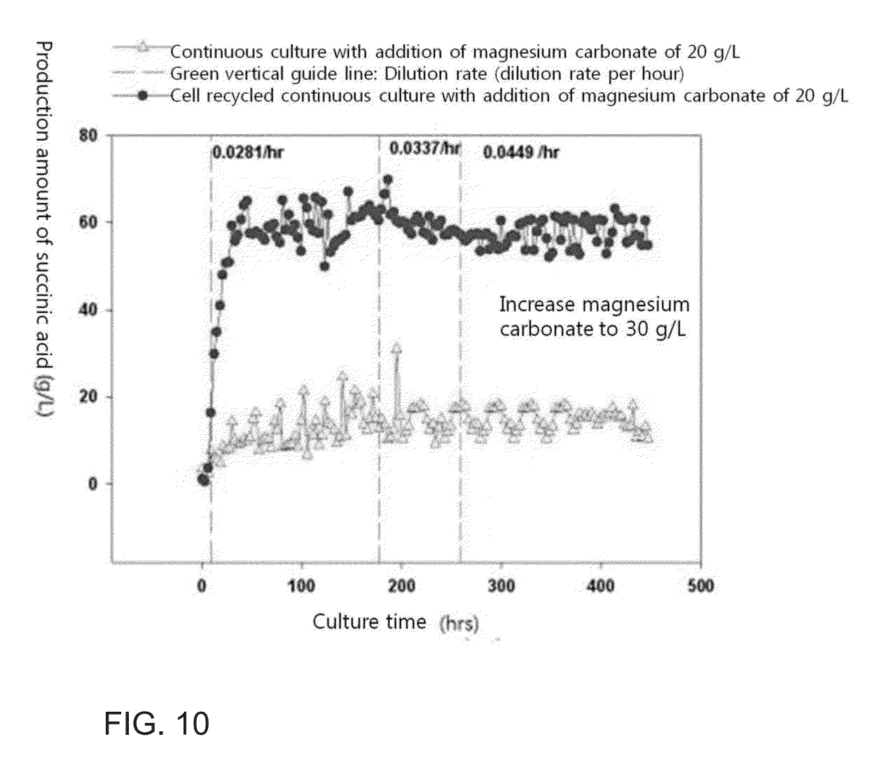

According to a result of analyzing a production amount of succinic acid in the continuous culture process with or without the cell recycled system, it could be observed that a production amount of succinic acid in the continuous culture was in the range of 12 to 18 g/L at a dilution rate of 0.281 l/hr in a steady state and an average production amount was about 14.65 g/L. Even when a dilution rate was increased to 0.0337 l/hr and 0.0449 l/hr, there was no big change in production amount of succinic acid, and an average production amount was about 14.7 g/L (FIG. 10, Table 1).

In the case of the continuous culture with addition of the cell recycled process, a production amount was 60.173 g/L at a dilution rate of 0.0281 l/hr, 60.580 g/L at a dilution rate of 0.0337 l/hr, and 57.459 g/L at a dilution rate of 0.0449 l/hr. As a dilution rate of microbial cells was increased, a production amount was also increased. There was a slight increase and a slight decrease in production amount of succinic acid, but there was no big difference. Therefore, it could be seen that a dilution rate or a concentration of the culture medium needed to be further increased.

4. Comparison in Yield Rate

In order to check efficiency of the continuous culture process with or without addition of the cell recycled process, a yield rate was calculated. A result of analyzing yield rates was as shown in Table 1 and FIGS. 4 and 5.

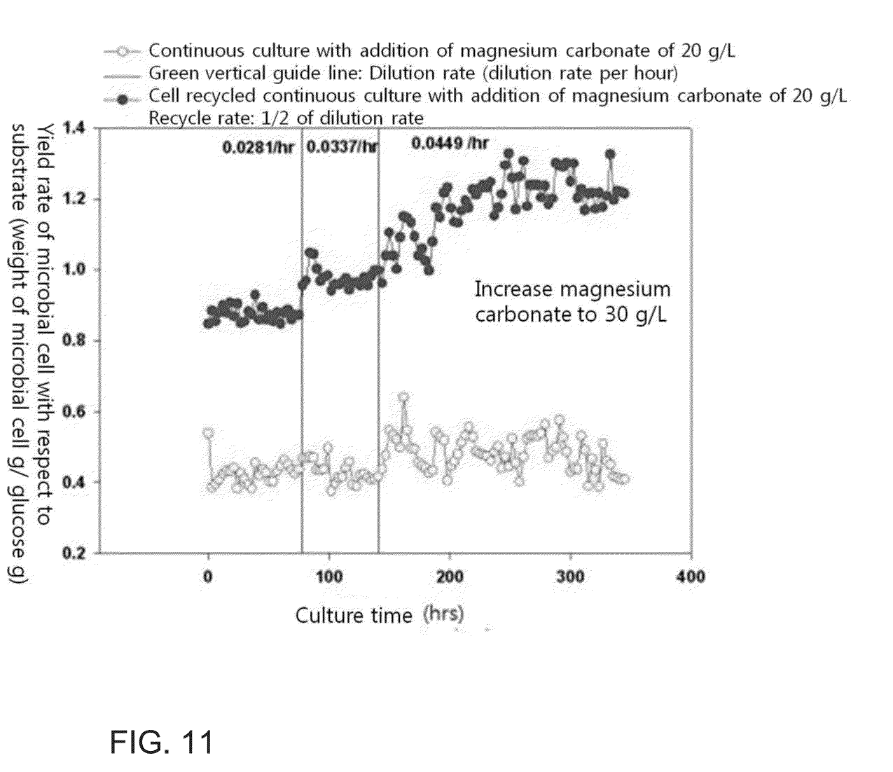

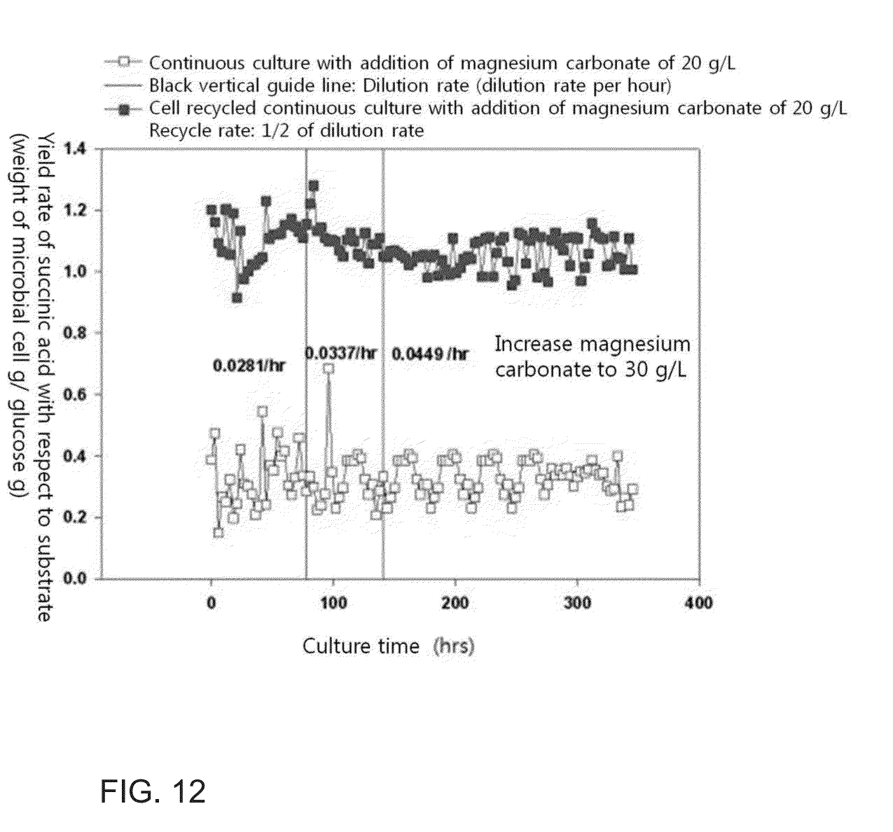

FIGS. 11 and 12 provide graphs comparing a yield rate of microbial cells with respect to glucose and a yield rate of succinic acid with respect to glucose by using the above-described information of an amount of microbial cells, an amount of residual glucose, and a production amount of succinic acid.

The analysis was carried out by calculating each sample of the graphs illustrated above. It was confirmed that a yield rate of microbial cells with respect to glucose in the cell recycled fermentation process was about 2 times higher overall than the continuous culture (refer to FIG. 11).

Further, it was confirmed from the graph that a yield rate of succinic acid with respect to glucose in the cell recycled fermentation process was about 4 to 5 times higher, and as a dilution rate was increased, the yield rate of succinic acid with respect to glucose remained almost unchanged (FIG. 12).

A result regarding the overall culture variables was as shown in Table 3. According to the analysis result, it could be seen that a yield rate of microbial cells with respect to glucose (Y_{X/S}: microbial cell (g)/glucose (g)) had the highest value of 1.196 at the highest dilution rate of 0.0449 l/hr in the cell recycled process, and in the case of addition of the cell recycled process, a yield rate was about 3 times higher.

Further, a yield rate of succinic acid with respect to glucose (Y_{P/S}: succinic acid (g)/glucose (g)) was not much different between the cell recycled processes but was about 3 times or higher than the continuous culture.

Furthermore, a yield rate of succinic acid with respect to amount of microbial cells (Y_{P/X}: succinic acid (g)/microbial cell (g)) had the highest value of 1.950 at the lowest dilution rate of 0.0281 l/hr in the cell recycled process, and in the case of addition of the cell recycled process, a yield rate was about 2 times higher.

TABLE 3
Culture Variables in Batch Culture, Continuous Culture, and Cell Recycled Process

| | Batch | | | | | | |
|---|---|---|---|---|---|---|---|
| Bioreactor | culture | Continuous | | | Recycled continuous | | |
| Dilution rate | — | 0.281 | 0.0337 | 0.0449 | 0.0281 (0.0141) | 0.0337 (0.0169) | 0.0449 (0.0225) |
| Steady X | 15.411 | 19.078 | 19.885 | 21.632 | 39.325 | 44.328 | 53.806 |
| Steady S | 0.000 | 0.284 | 0.000 | 0.000 | 0.000 | 0.000 | 0.000 |
| Steady P | 32.343 | 14.650 | 14.632 | 14.924 | 60.173 | 60.025 | 57.459 |
| qs | 0.034 | 0.659 | 0.076 | 0.093 | 0.048 | 0.051 | 0.056 |
| qp | 0.024 | 0.022 | 0.026 | 0.031 | 0.064 | 0.073 | 0.072 |
| γX | 0.321 | 0.536 | 0.719 | 0.972 | 1.301 | 1.861 | 3.185 |
| γS | 0.517 | 1.257 | 1.614 | 2.022 | 1.531 | 1.531 | 1.531 |
| DP | 0.372 | 0.412 | 0.523 | 0.671 | 2.536 | 3.221 | 3.873 |
| Y_{X/S} | 0.342 | 0.427 | 0.442 | 0.481 | 0.874 | 0.985 | 1.196 |
| Y_{P/S} | 0.719 | 0.328 | 0.325 | 0.332 | 1.104 | 1.101 | 1.054 |
| Y_{P/X} | 2.099 | 0.772 | 0.740 | 0.695 | 1.950 | 1.739 | 1.230 |

The abbreviations in Table 3 have the meanings as follows.
Batch culture: Batch culture
Continuous culture: Continuous culture
Recycled continuous: Cell recycled continuous culture
Steady X: Amount of microbial cells in a steady state in a bioreactor (g/L)
Steady S: Amount of residual glucose in a steady state in a bioreactor (g/L)
Steady P: Production amount of succinic acid in a steady state in a bioreactor (g/L)
qs: Consumption rate of glucose with respect to cell (glucose (g)/cell (g)/hour)
qp: Production rate of succinic acid with respect to cell (succinic acid (g)/cell (g)/hour)
γX: Microbial cell growth velocity (amount of microbial cell (g)/liter/hour)
γS: Consumption rate of glucose (glucose (g)/liter/hour)
DP: Production velocity of succinic acid (succinic acid (g)/liter/hour)
Y_{X/S}: Yield rate of microbial cells with respect to glucose (microbial cell (g)/glucose (g))
Y_{P/S}: Yield rate of succinic acid with respect to glucose (succinic acid (g)/glucose (g))
Y_{P/X}: Yield rate of succinic acid with respect to amount of microbial cells (succinic acid (g)/microbial cell (g))

As the calculation result of the yield rates, it could be seen that the continuous culture with addition of the cell recycled process had the higher yield rates overall than the continuous culture without addition of the cell recycled process, and efficiently used the supplied culture medium and quantitatively mass-produce succinic acid.

While the present invention has been shown and described with reference to preferable Examples thereof, it will be understood by those skilled in the art that various changes in form and details may be made therein without departing from the spirit and scope of the present invention as defined by the appended claims. Therefore, the disclosed Examples should not be considered in view of explanation, but no limitation. The technical scope of the present invention is taught in the claims, but not the detailed description, and all the differences in the equivalent scope thereof should be construed as falling within the present invention.

What is claimed is:

1. A continuous culture method for mass production of succinic acid using Actinobacillus succinogenes, the continuous culture method comprising:
continuously supplying a culture medium comprised of glucose, yeast extract, and corn steep liquor, and sodium hydrogen carbonate (NaHCO₃) to a bioreactor; and
continuously removing a culture fluid from the bioreactor.

2. The continuous culture method of claim 1, further comprising:
separating the Actinobacillus succinogenes from the continuously removed culture fluid; and
supplying the separated Actinobacillus succinogenes to the bioreactor.

3. The continuous culture method of claim 1, further comprising:
supplying magnesium carbonate (MgCO₃) having a concentration of 10 to 30 g/L at a velocity of 2 to 4 ml/hr.

4. The continuous culture method of claim 1, wherein the culture medium is supplied at a velocity of 1 to 100 ml/hr and the sodium hydrogen carbonate (NaHCO₃) having a concentration of 8 to 10 g/L is supplied at a velocity of 2 to 4 ml/hr.

5. The continuous culture method of claim 1, wherein the continuous culture method aerates and supplies carbon dioxide at a speed of 0.4 to 0.8 vvm.

6. The continuous culture method of claim 1, wherein the glucose is contained at a concentration of 45 to 65 g/L, the yeast extract is contained at a concentration of 5 to 8 g/L, and the corn steep liquor is contained at a concentration of 1 to 14 g/L.

7. The continuous culture method of claim 1, wherein the Actinobacillus succinogenes is a strain UK13 (KCTC 12233BP).

8. The continuous culture method of claim 2, wherein the culture medium, sodium hydrogen carbonate, and magnesium carbonate are supplied at a velocity of 1 to 100 ml/hr.

9. The continuous culture method of claim 2, further comprising:
increasing a concentration of microbial cells by supplying the continuously removed culture fluid to the bioreactor at a velocity of 15 to 22.5 ml/hr.

10. The continuous culture method of claim 2, wherein the step of separating the Actinobacillus succinogenes is carried out by using a cell separator.

11. The continuous culture method of claims 1, wherein the culture medium, the sodium hydrogen carbonate, or the magnesium carbonate are supplied by using a drip tube.

12. A continuous culture system for mass production of succinic acid using Actinobacillus succinogenes, the continuous culture system comprising:
a bioreactor in which a culture fluid is stored;
a cell separator configured to separate a culture fluid supplied to the bioreactor into microbial cells and the culture fluid and discharge the microbial cells and the culture fluid;
a drip tube configured to prevent contamination of a culture medium supplied from a culture medium supply unit and supply the culture medium to the bioreactor while controlling a supply rate of the culture medium; and
a collection line configured to collect the microbial cells discharged from the cell separator and supply the microbial cells to the bioreactor.

13. The continuous culture system of claim 12, wherein the cell separator has a predetermined size and comprises an upper vertical portion formed at an upper part, a culture fluid outlet having a smaller diameter than the upper vertical portion and provided under the upper vertical portion with a space between them, and an inclined portion is formed between the upper vertical portion and the culture fluid outlet.

14. The continuous culture system of claim 12, wherein the cell separator has a predetermined size and comprises an upper inclined portion formed at an upper part, a culture fluid outlet having a smaller diameter than the upper inclined portion and provided under the upper inclined portion with a space between them, and an upper vertical connection portion and a lower inclined connection portion are continuously formed between the upper inclined portion and the culture fluid outlet.

15. The continuous culture system of claim 13, wherein an inner surface of the cell separator includes a curved surface guiding portion that allows the culture fluid to be rapidly discharged through the culture fluid outlet.

16. The continuous culture system of claim 12, wherein the cell separator comprises a cylindrical tube or a filter comprised of multiple mesh layers configured to separate the culture fluid supplied to the bioreactor into microbial cells and the culture fluid.

17. The continuous culture system of claim 12, wherein the drip tube includes a tube connection portion connected to the culture medium supplying unit and a speed control unit configured to control a supply rate of the culture medium.

18. The continuous culture system of claim 12, wherein at one side of the drip tube, a carbon dioxide inlet is formed to supply carbon dioxide accommodated therein to the inside or a predetermined position.
